(19) United States
(12) Patent Application Publication
(10) Pub. No.: US 2013/0236941 A1
(43) Pub. Date: Sep. 12, 2013

(54) Integrated Biorefinery

(71) Applicant: COBALT TECHNOLOGIES INC., Mountain View, CA (US)

(72) Inventors: Stacy M. Burns-Guydish, Campbell, CA (US); Anthony F. Cann, San Francisco, CA (US); Dhruti Dalal, Sunnyvale, CA (US); Lawrence W. Fry, Palo Alto, CA (US); Michael S. Hershkowitz, San Jose, CA (US); William F. McDonald, Utica, OH (US); Andrew D. Meyer, Aurora, IL (US); Victor O. Nava-Salgado, Cupertino, CA (US); Brandon T. Olson, Mountain View, CA (US); Ishmael M. Sonico, Aliso Viejo, CA (US); David C. Walther, Oakland, CA (US); Yongming Zhu, San Jose, CA (US)

(73) Assignee: COBALT TECHNOLOGIES INC., Mountain View, CA (US)

(21) Appl. No.: 13/794,741

(22) Filed: Mar. 11, 2013

Related U.S. Application Data
(60) Provisional application No. 61/609,935, filed on Mar. 12, 2012.
(60) Provisional application No. 61/619,532, filed on Apr. 3, 2012.

(51) Int. Cl. C12M 1/00; C12N 1/20; C12P 7/10; C12P 7/16; C12P 7/28

(52) U.S. Cl. CPC C12M 45/06; C12M 45/00; C12M 45/20; C12P 7/16; C12P 7/28; C12P 7/10; C12N 1/20
USPC 435/165; 435/289.1; 435/160; 435/150; 435/253.6

(57) ABSTRACT

Systems and methods for producing bioproducts are shown. The systems and methods herein can be configured and used in an integrated biorefinery. The integrated biorefinery may comprise a sugar production facility such as a sugar mill, a production facility for one or more bioproduct(s) such as butanol, and optionally an ethanol production facility employing the system and method.

### CROSS-REFERENCE TO RELATED APPLICATIONS

This application claims the benefit of U.S. Application No. 61/609,935, filed on Mar. 12, 2012, and U.S. Application No. 61/619,532, filed on Apr. 3, 2012, which are incorporated herein by reference in their entireties.

### FIELD

The present inventions relate to a bioproduct (e.g., butanol) production facility.

The present inventions also relate to a bioproduct (e.g., butanol) production facility that is configured to produce one or more bioproduct(s) (e.g., butanol) from biomass.

The present inventions further relate to a bioproduct (e.g., butanol) production facility that can be integrated with a sugar production facility (e.g., a sugar mill).

The present inventions further relate to a bioproduct (e.g., butanol) production facility that can be co-located adjacent to and integrated with a sugar production facility (e.g., a sugar mill) and an ethanol production facility.

The present inventions further relate to a bioproduct (e.g., butanol) production facility that is configured so that matter (e.g., resources such as residual biomass, by-products, process streams, other available material, etc.) from a sugar production facility (e.g., sugar mill) and/or ethanol production facility can be supplied to the bioproduct (e.g., butanol) production facility and used to facilitate the production of one or more bioproduct(s) (e.g., butanol).

### BACKGROUND

Systems and methods for the production of butanol are generally known. Such systems and methods are implemented in a butanol production facility.

Systems and methods for the production of ethanol are generally known. Such systems and methods are implemented in an ethanol production facility.

Systems and methods for the production of sugar and related products from sugar-containing plant matter (e.g., sugar cane, sugar beets, etc.) are generally known. Such systems and methods are typically implemented in a sugar production facility or sugar mill. The processing of sugar cane into sugar and other products results in the availability of (among other things) residual biomass such as bagasse or sugar beet pulp.

It is also generally known to locate an ethanol production facility adjacent to a sugar mill. In known arrangements, the sugar mill supplies sugar-containing matter (e.g., sugars and molasses) for the fermentation process of the ethanol production facility; ethanol is recovered from the fermentation broth. The production of ethanol from sugar cane results in the availability of (among other things) residual process streams such as vinasse.

### SUMMARY

It would be advantageous to configure a bioproduct (e.g., butanol) production facility to produce one or more bioproduct(s) (e.g., butanol) from biomass, for example, in a microbial fermentative process that converts the biomass (e.g., sugars and/or nutrients derived from the biomass) to bioproduct(s).

It would also be advantageous to integrate a bioproduct (e.g., butanol) production facility with a sugar production facility (e.g., a sugar mill), for example, a facility in which sugar molecules are extracted from sugar cane, so that residual biomass such as bagasse could be supplied to the bioproduct (e.g., butanol) production facility as a feedstock. For example, sugar molecules may be extracted from such a feedstock, such as bagasse, to support a microbial fermentation process for production of one or more bioproduct(s) (e.g., butanol), for example, serving as a carbon source for such a fermentation.

It would also be advantageous to integrate a bioproduct (e.g., butanol) production facility with a sugar production facility (e.g., a sugar mill), for example, a facility in which sugar molecules are extracted from sugar cane, so that such extracted sugar molecules (e.g., cane juice, molasses) could be supplied to the bioproduct (e.g., butanol) production facility to support production of one or more bioproduct(s) (e.g., butanol), for example, serving as a carbon source for a microbial fermentation process for production of the bioproduct(s).

It would further be advantageous to integrate a bioproduct (e.g., butanol) production facility with an ethanol production facility, e.g., a sugar cane ethanol production facility, for example, so that residual process streams from the ethanol production facility, such as vinasse, could be supplied to the bioproduct (e.g., butanol) production facility and used in the processing of feedstocks and/or sugar molecules into bioproduct(s) (e.g., butanol), for example, in a microbial fermentation process for production of the bioproduct(s).

In one aspect, an integrated biorefinery is provided. An integrated biorefinery as disclosed herein includes: (a) a sugar production facility; and (b) a bioproduct production facility. In the sugar production facility, sugar-containing biomass (e.g., sugar cane and/or sorghum) is processed to extract sugar, thereby producing a liquid sugar-containing extract and residual bagasse. The bioproduct production facility includes at least one microorganism that is capable of producing at least one bioproduct of interest in a microbial fermentation process. Sugar molecules that are extracted from sugar-containing biomass (e.g., sugar cane) that is processed in the sugar processing facility are provided to the microorganism in a growth medium. The sugar molecules are fermented by the microorganism, thereby producing a fermentation broth that contains at least one bioproduct of interest. In one embodiment, the microorganism(s) in the bioproduct production facility include at least one Clostridium strain.

In some embodiments, the sugar molecules that are provided to the microorganism in the bioproduct production facility include: (i) at least a portion of the liquid sugar-containing extract that is produced in the sugar production facility, and (ii) sugar molecules that are extracted from at least a portion of the residual bagasse from the sugar production facility and/or sugar molecules extracted from at least a portion of biomass (e.g., cane straw) that is removed from the sugar-containing biomass (e.g., sugar cane) prior to processing in the sugar production facility.

In some embodiments, the liquid sugar-containing extract may include cane juice and/or molasses.

In some embodiments, the sugar molecules that are extracted from at least a portion of the bagasse and/or biomass that is removed prior to sugar processing (e.g., cane straw) are extracted by acid hydrolysis, thereby producing a liquid hydrolysate that comprises soluble sugar molecules and residual solid material. In some embodiments, the hydrolysate includes C5 sugar molecules. In one embodiment, nitric acid is used for acid hydrolysis. The residual solid material from the acid hydrolysis may optionally be separated from the liquid hydrolysate. In one embodiment, the residual solid material is provided to a boiler as a fuel source. Optionally, at least a portion of nitrates that are present in the residual solid material may be removed prior to use of the material in the boiler. In some embodiments, the residual solid material from the acid hydrolysis may include cellulose. The residual cellulose-containing material may optionally be treated to extract sugar molecules from the cellulose. For example, extraction of sugar molecules from cellulose may include treatment with at least one enzyme that that catalyzes hydrolysis of cellulose, e.g., at least one cellulase enzyme, thereby producing a liquid enzymatic hydrolysate that comprises soluble sugar molecules and a second residual solid material. In one embodiment, the second residual solid material is provided to a boiler as a fuel source. Optionally, at least a portion of nitrates that are present in the second residual solid material may be removed prior to use of the material in the boiler.

In some embodiments, at least one bioproduct is separated from the fermentation broth in the bioproduct production facility, thereby producing vinasse. At least a portion of the vinasse may be recycled and provided to the bioproduct production facility as liquid in the growth medium for further production of bioproduct(s). In one embodiment, butanol is separated from the fermentation broth and the vinasse is butanol vinasse.

In some embodiments, at least one solvent may be produced in the bioproduct production facility, for example, butanol and/or acetone. In one embodiment, butanol is produced in the bioproduct production facility. In another embodiment, acetone is produced in the bioproduct production facility. In another embodiment, butanol and acetone are produced in the bioproduct production facility.

In some embodiments, processing of sugar-containing biomass (e.g., sugar cane) in the sugar production facility includes steam. In one embodiment, steam condensate may be produced from the steam, and at least a portion of the steam condensate may provided to the bioproduct production facility as liquid in the growth medium for production of the bioproduct(s). In another embodiment, at least a portion of the steam may be recovered and used to provide heat for the fermentation process in the bioproduct production facility.

In some embodiments, the integrated biorefinery further includes: (c) an ethanol production facility. The ethanol production facility includes at least one second microorganism that is capable of producing ethanol in a second microbial fermentation process. Sugar molecules that are extracted from sugar-containing biomass (e.g., sugar cane and/or sorghum) that is processed in the sugar production facility are provided to the second microorganism(s) in a second growth medium. The sugar molecules are fermented by the second microorganism(s), thereby producing a second fermentation broth that contains ethanol. In some embodiments, ethanol is separated from the second fermentation broth, thereby producing ethanol vinasse. The ethanol vinasse may be provided to the bioproduct production facility as liquid for the growth medium. In some embodiments, the ethanol vinasse provides nutrients for growth of the microorganism in the bioproduct production facility.

In some embodiments, the second microorganism(s) in the ethanol production facility includes yeast. In one embodiment, at least a portion of the yeast from the ethanol production facility may be provided to the bioproduct production facility in the growth medium as nutrition for the growth of the microorganism(s) and/or production of bioproduct(s). In another embodiment, at least a portion of the yeast from the ethanol production facility is added during acid hydrolysis of sugar-containing biomass (e.g., bagasse and/or cane straw) to produce a biomass hydrolysate that includes hydrolyzed yeast cells. The hydrolysate may be provided in the growth medium for the microorganism(s) in the bioproduct production facility, and the yeast may provide nutrients for growth of the microorganism(s) and/or production of bioproduct(s).

In some embodiments, spent microorganisms from the fermentation in the bioproduct production facility may be recovered for other uses, and processed for incorporation into other products. In one embodiment, microorganisms from the bioproduct production facility are incorporated into an animal feed product. In other embodiments, microorganisms from the bioproduct production facility are used as a soil amendment and/or a roadway amendment.

In another aspect, a process for producing at least one bioproduct is provided. The process includes culturing one or more microorganism(s) that are capable of producing the bioproduct(s) in a growth medium that contains a liquid sugar-containing extract from processing of sugar-containing biomass (e.g., sugar cane and/or sorghum) and sugar molecules extracted from biomass that remains after sugar processing (e.g., bagasse) and/or biomass material that is removed prior to sugar processing (e.g., cane straw). In some embodiments, the liquid sugar-containing extract includes cane juice and/or molasses. In some embodiments, the sugar molecules that are extracted from biomass (e.g., bagasse and/or cane straw) are extracted by acid hydrolysis. In some embodiments, the sugar molecules that are extracted from biomass (e.g., bagasse and/or cane straw) are extracted by a combination of acid hydrolysis and enzymatic hydrolysis. In some embodiments, the growth medium further includes vinasse (e.g., bioproduct (e.g., butanol) vinasse and/or ethanol vinasse). In some embodiments, the bioproduct(s) includes at least one solvent, such as butanol and/or acetone. In some embodiments, the microorganism(s) includes at least one Clostridium strain.

In another aspect, a process for producing at least one bioproduct is provided. The process includes: (a) processing sugar-containing biomass (e.g., sugar cane and/or sorghum) in a sugar production facility, thereby producing a liquid sugar-containing extract and residual bagasse; and (b) culturing at least one microorganism in a bioproduct production facility, wherein sugar molecules that are extracted from sugar-containing biomass (e.g., sugar cane) that is processed in the sugar production facility are provided to the microorganism(s) in a growth medium, wherein the sugar molecules are fermented by the microorganism(s), thereby producing a fermentation broth that includes said at least one bioproduct. In some embodiments, the microorganism(s) in the bioproduct production facility include at least one Clostridium strain.

In some embodiments, the sugar molecules that are provided to the microorganism in the bioproduct production facility include: (i) at least a portion of the liquid sugar-containing extract that is produced in the sugar production facility, and (ii) sugar molecules that are extracted from at least a portion of the residual bagasse from the sugar production facility and/or sugar molecules extracted from at least a portion of biomass (e.g., cane straw) that is removed from the sugar-containing biomass (e.g., sugar cane) prior to processing in the sugar production facility.

In some embodiments, the liquid sugar-containing extract may include cane juice and/or molasses.

In some embodiments, the sugar molecules that are extracted from at least a portion of the bagasse and/or biomass that is removed prior to sugar processing (e.g., cane straw) are extracted by acid hydrolysis, thereby producing a liquid hydrolysate that comprises soluble sugar molecules and residual solid material. In some embodiments, the hydrolysate includes C5 sugar molecules. In one embodiment, nitric acid is used for acid hydrolysis. The residual solid material from the acid hydrolysis may optionally be separated from the liquid hydrolysate. In one embodiment, the residual solid material is provided to a boiler as a fuel source. Optionally, at least a portion of nitrates that are present in the residual solid material may be removed prior to use of the material in the boiler. In some embodiments, the residual solid material from the acid hydrolysis may include cellulose. The residual cellulose-containing material may optionally be treated to extract sugar molecules from the cellulose. For example, extraction of sugar molecules from cellulose may include treatment with at least one enzyme that that catalyzes hydrolysis of cellulose, e.g., at least one cellulase enzyme, thereby producing a liquid enzymatic hydrolysate that comprises soluble sugar molecules and a second residual solid material. In one embodiment, the second residual solid material is provided to a boiler as a fuel source. Optionally, at least a portion of nitrates that are present in the second residual solid material may be removed prior to use of the material in the boiler.

In some embodiments, at least one bioproduct is separated from the fermentation broth in the bioproduct production facility, thereby producing vinasse. At least a portion of the vinasse may be recycled and provided to the bioproduct production facility as liquid in the growth medium for further production of bioproduct(s). In one embodiment, butanol is separated from the fermentation broth and the vinasse is butanol vinasse.

In some embodiments, at least one solvent may be produced in the bioproduct production facility, for example, butanol and/or acetone. In one embodiment, butanol is produced in the bioproduct production facility. In another embodiment, acetone is produced in the bioproduct production facility. In another embodiment, butanol and acetone are produced in the bioproduct production facility.

In some embodiments, processing of sugar-containing biomass (e.g., sugar cane) in the sugar production facility includes steam. In one embodiment, steam condensate may be produced from the steam, and at least a portion of the steam condensate may provided to the bioproduct production facility as liquid in the growth medium for production of the bioproduct(s). In another embodiment, at least a portion of the steam may be recovered and used to provide heat for the fermentation process in the bioproduct production facility.

In some embodiments, the process for producing at least one bioproduct further includes: (c) producing ethanol in an ethanol production facility. The ethanol production facility includes at least one second microorganism that is capable of producing ethanol in a second microbial fermentation process. Sugar molecules that are extracted from sugar-containing biomass (e.g., sugar cane and/or sorghum) that is processed in the sugar production facility are provided to the second microorganism(s) in a second growth medium. The sugar molecules, are fermented by the second microorganism(s), thereby producing a second fermentation broth that includes ethanol. In some embodiments, ethanol is separated from the second fermentation broth, thereby producing ethanol vinasse. The ethanol vinasse may be provided as liquid in the growth medium for the microorganism in the bioproduct production facility. In some embodiments, the ethanol vinasse provides nutrients for growth of the microorganism in the bioproduct production facility.

In some embodiments, the second microorganism(s) in the ethanol production facility includes yeast. In one embodiment, at least a portion of the yeast from the ethanol production facility may be provided to the bioproduct production facility in the growth medium as nutrition for the growth of the microorganism(s) and/or production of bioproduct(s). In another embodiment, at least a portion of the yeast from the ethanol production facility is added during acid hydrolysis of sugar-containing biomass (e.g., bagasse and/or cane straw) to produce a biomass hydrolysate that includes hydrolyzed yeast cells. The hydrolysate may be provided in the growth medium for the microorganism(s) in the bioproduct production facility, and the yeast may provide nutrients for growth of the microorganism(s) and/or production of bioproduct(s).

In some embodiments, spent microorganisms from the fermentation in the bioproduct production facility may be recovered for other uses, and processed for incorporation into other products. In one embodiment, microorganisms from the bioproduct production facility are incorporated into an animal feed product. In other embodiments, microorganisms from the bioproduct production facility are used as a soil amendment and/or a roadway amendment.

In another aspect, a growth medium for culturing a microorganism is provided. The growth medium includes: (a) a liquid sugar-containing extract from processing of sugar-containing biomass (e.g., sugar cane and/or sorghum) in a sugar production facility; and (b) sugar molecules extracted from biomass remaining after sugar processing (e.g., bagasse) and/or biomass removed prior to sugar processing (e.g., cane straw). In one embodiment, the liquid sugar-containing extract includes cane juice and/or molasses. In one embodiments, sugar molecules extracted from biomass remaining after sugar processing (e.g., bagasse) and/or biomass removed prior to sugar processing (e.g., cane straw) are extracted by acid hydrolysis. In another embodiment, sugar molecules extracted from biomass remaining after sugar processing (e.g., bagasse) and/or biomass removed prior to sugar processing (e.g., cane straw) are extracted by a combination of acid hydrolysis and enzymatic hydrolysis. In some embodiments, the growth medium further includes vinasse. In one embodiment, the growth medium further includes steam condensate from a sugar production facility. In some embodiments, the growth medium further includes hydrolyzed and/or lysed yeast cells, e.g., spent yeast cells from an ethanol production facility.

### BRIEF DESCRIPTION OF THE DRAWINGS

FIG. 1 is a schematic diagram of a combined sugar and ethanol production facility according to an exemplary embodiment.

FIG. 2A is a schematic block diagram of an integrated biorefinery according to an exemplary embodiment.

FIG. 2B is an exemplar functional site layout of a butanol production facility according to an exemplary embodiment.

FIG. 3A is a block flow diagram of an integrated biorefinery according to an exemplary embodiment.

FIG. 3B is a block flow diagram of an integrated biorefinery according to an exemplary embodiment.

FIG. 4A is a schematic diagram of an integrated biorefinery according to an exemplary embodiment.

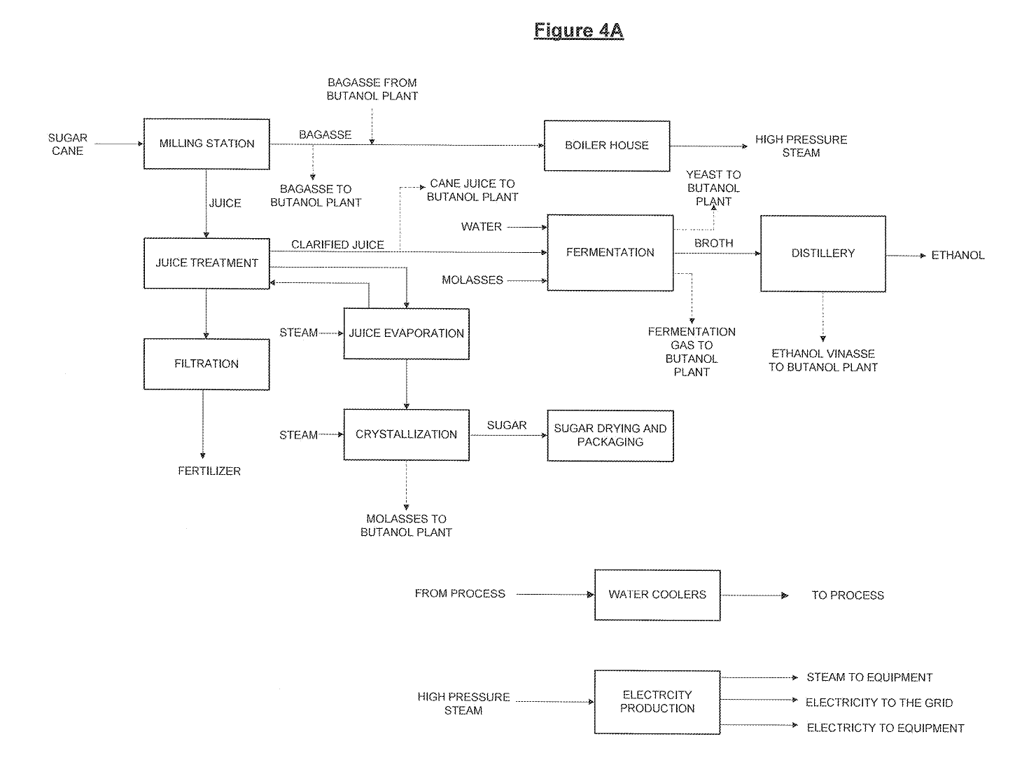

FIG. 4B is a schematic diagram of an integrated biorefinery according to an exemplary embodiment.

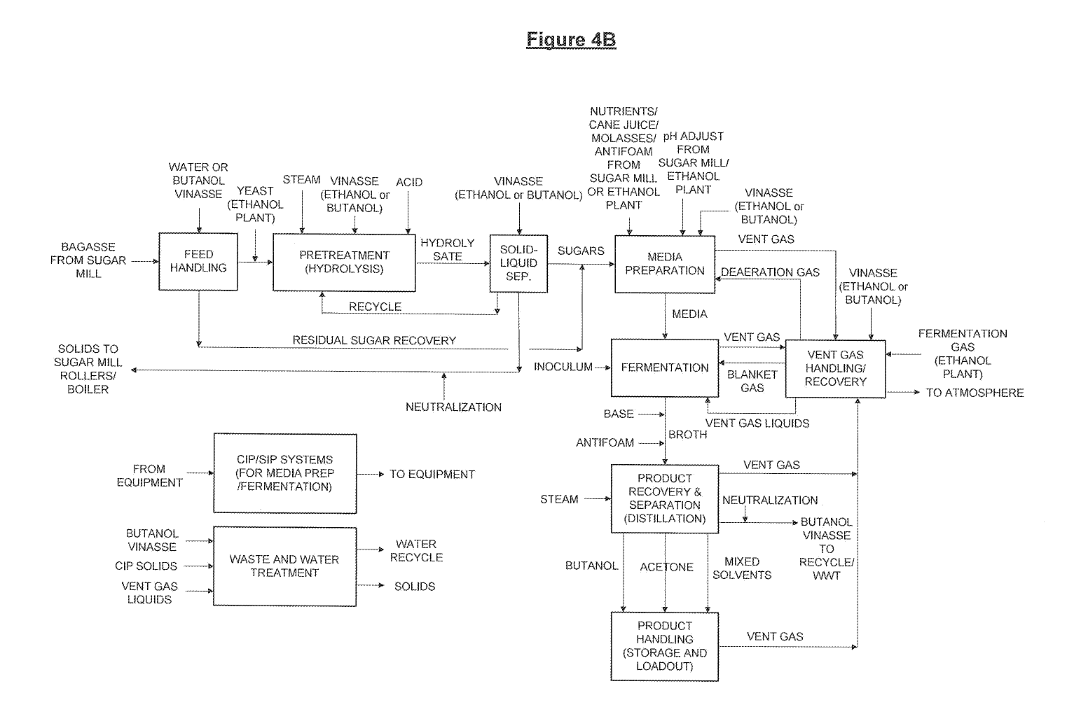

FIG. 5A is a block flow diagram of butanol production facility according to an exemplary embodiment.

FIG. 5B is a block flow diagram of an integrated biorefinery according to an exemplary embodiment.

FIG. 6A is a block flow diagram of an integrated biorefinery according to an exemplary embodiment.

FIG. 6B is a block flow diagram of a milling system for an integrated biorefinery according to an exemplary embodiment.

FIG. 6C is a block flow diagram of a pre-fermentation conditioning system for a butanol production facility according to an exemplary embodiment.

FIG. 6D is a block flow diagram of a distillation system for a butanol production facility according to an exemplary embodiment.

FIG. 7A is a schematic diagram of a milling system for a sugar production facility according to an exemplary embodiment.

FIG. 7B is a schematic diagram of a juice treatment system for a sugar production facility according to an exemplary embodiment.

FIG. 7C is a schematic diagram of a crystallization and packing system for a sugar production facility according to an exemplary embodiment.

FIG. 7D is a schematic diagram of a fermentation system for an ethanol production facility according to an exemplary embodiment.

FIG. 7E is a schematic diagram of a distillation system for an ethanol production facility according to an exemplary embodiment.

FIG. 7F is a schematic diagram of utility systems for an ethanol production facility according to an exemplary embodiment.

FIG. 8A is a schematic diagram of pre-treatment and media preparation systems for a butanol production facility according to an exemplary embodiment.

FIG. 8B is a schematic diagram of a fermentation system for a butanol production facility according to an exemplary embodiment.

FIG. 8C is a schematic diagram of product recovery and storage systems for a butanol production facility according to an exemplary embodiment.

### DETAILED DESCRIPTION

### Definitions

“A,” “an” and “the” include plural references unless the context clearly dictates otherwise.

“Bioproduct” refers to any substance of interest produced biologically, i.e., via a metabolic pathway, by a microorganism, e.g., in a microbial fermentation process. Bioproducts include, but are not limited to biofuels, solvents, biomolecules (e.g., proteins (e.g., enzymes), polysaccharides), organic acids (e.g., formate, acetate, butyrate, propionate, succinate), alcohols (e.g., methanol, propanol, isopropanol, hexanol, 2-butanol, isobutanol), diols (e.g., 1,3-propanediol), fatty acids, aldehydes, lipids, long chain organic molecules (for example, for use in surfactant production), vitamins, and sugar alcohols (e.g., xylitol).

“Biofuel” refers to fuel molecules (e.g., n-butanol, acetone, ethanol, isobutanol, farnesene, etc.) produced biologically by a microorganism, e.g., in a microbial fermentation process.

“Byproduct” refers to a substance that is produced and/or purified and/or isolated during any of the processes described herein, which may have economic or environmental value, but that is not the primary process objective. Nonlimiting examples of byproducts of the processes described herein include lignin compounds and derivatives, carbohydrates and carbohydrate degradation products (e.g., furfural, hydroxymethyl furfural, formic acid), and extractives (described infra).

“Feedstock” refers to a substance that can serve as a source of sugar molecules to support microbial growth in a fermentation process.

“Deconstruction” refers to mechanical, chemical, and/or biological degradation of biomass to render individual components (e.g., cellulose, hemicellulose) more accessible to further pretreatment processes, for example, a process to release monomeric and oligomeric sugar molecules, such as acid hydrolysis.

“Conditioning” refers to removal of inhibitors of microbial growth and/or bioproduct, e.g., biofuel, production from a hydrolysate produced by hydrolysis of a cellulosic feedstock or adjustment of a physical parameter of the hydrolysate to render it more amenable to inclusion in a microbial culture medium, for example, adjustment of the pH to a pH that is suitable for growth of the microorganism when added to a microbial growth medium.

“Titer” refers to amount of a substance produced by a microorganism per unit volume in a microbial fermentation process. For example, biobutanol titer may be expressed as grams of butanol produced per liter of solution.

“Yield” refers to amount of a product produced from a feed material (for example, sugar, relative to the total amount that of the substance that would be produced if all of the feed substance were converted to product. For example, butanol yield may be expressed as % of butanol produced relative to a theoretical yield if 100% of the feed substance (for example, sugar) were converted to biobutanol.

“Productivity” refers to the amount of a substance produced by a microorganism per unit volume per unit time in a microbial fermentation process. For example, butanol productivity may be expressed as grams of butanol produced per liter of solution per hour.

“Sugar conversion” refers to grams of sugar consumed by a microorganism (e.g., in a microbial fermentation process) per grams of sugar provided to the microorganism (e.g., grams of sugar provided in a microbial growth medium).

“Wild-type” refers to a microorganism as it occurs in nature.

“ABE fermentation” refers to production of acetone, butanol, and/or ethanol by a fermenting microorganism.

“Lignocellulosic” biomass refers to plant biomass that contains cellulose, hemicellulose, and lignin. The carbohydrate polymers (cellulose and hemicellulose) are tightly bound to lignin.

“Lignins” are macromolecular components of lignocellulosic biomass that contain phenolic propylbenzene skeletal units linked at various sites.

“Solvent” refers to a liquid or gas that is capable of dissolving a solid or another liquid or gas. A solvent may be produced as a bioproduct by a microorganism as described herein. Nonlimiting examples of solvents produced by microorganisms include n-butanol, acetone, ethanol, acetic acid, isopropanol, n-propanol, methanol, formic acid, 1,4-dioxane, tetrahydrofuran, acetonitrile, dimethylformamide, and dimethyl sulfoxide.

n-Butanol is also referred to as “butanol” herein.

“Direct steam” refers to steam that is added into a process stream.

“Indirect steam” refers to steam that is not in direct contact with a process fluid, for example, steam that is injected into a jacket or heat exchanger.

“Vinasse” refers to a fermentation broth from which one or more bioproduct has been removed. For example, fermentation broth of a microorganism that produces ethanol and from which ethanol has been removed is termed “ethanol vinasse.” As a further example, fermentation broth of a microorganism that produces butanol and from which butanol has been removed is termed “butanol vinasse.” In some embodiments, vinasse the bottom fraction of distillation of a solvent fermentation process, and solvent and other volatile compounds are separated from the fermentation broth while the rest of the constituents (e.g., residual sugar, organic acids, glycerol, biomass) are slightly concentrated in the vinasse.

“Aerotolerant” refers to a microorganism that is able to grow in the presence of O₂.

“Plant” and “facility” are used interchangeably herein to describe a location and equipment in which a disclosed process (e.g., sugar cane processing, ethanol production, bioproduct production) occurs.

### Integrated Biorefinery System

An integrated biorefinery system is provided for production of one or more bioproduct(s) of interest. The system includes a sugar production facility and a bioproduct production facility. The sugar production facility and the bioproduct production facility are integrated, such that process streams and/or residual materials from the sugar production facility are utilized to support production of the bioproduct(s). In some embodiments, the bioproduct production facility is configured to receive materials (e.g., residual biomass, such as bagasse and/or cane straw) and/or process streams (e.g., cane juice and/or molasses; steam; steam condensate) from the sugar production facility, and/or to provide materials (e.g., residual solid material from biomass pretreatment, for example for use as a fuel) and/or process streams to the sugar processing facility. In some embodiments, the bioproduct production facility is in fluid communication with the sugar production facility. In some embodiments, the bioproduct production facility is co-located (e.g., adjacent and/or in close physical proximity) with the sugar production facility.

In some embodiments, the integrated biorefinery system further includes an ethanol production facility. In such embodiments, the sugar production facility, the ethanol production facility, and the bioproduct production facility are integrated, such that process streams and/or residual materials from the sugar production facility are utilized to support production of ethanol and the bioproduct(s), and process streams and/or residual materials from the ethanol production facility are utilized to support production of the bioproduct(s). In some embodiments, the bioproduct production facility is configured to receive materials (e.g., residual biomass, such as bagasse and/or cane straw) and/or process streams (e.g., cane juice and/or molasses; steam; steam condensate) from the sugar production facility, and/or to receive materials (e.g., killed yeast cells) and/or process streams (e.g., vinasse; fermentation gas) from the ethanol production facility, and/or to provide materials (e.g., residual solid material from biomass pretreatment, for example for use as a fuel) and/or process streams to the sugar processing facility. In some embodiments, the bioproduct production facility is in fluid communication with the ethanol production facility and with the sugar production facility. In some embodiments, the bioproduct production facility, the ethanol production facility, and the sugar production facility are co-located (e.g., adjacent and/or in close physical proximity).

Sugar-containing biomass, e.g., sugar cane and/or sorghum, may be processed in a sugar production facility to extract sugar, e.g., sucrose. Sugar may be extracted in the form of sugar-containing liquid(s) (e.g., cane juice; molasses), thereby producing sugar-containing liquid stream(s) and residual biomass (e.g., bagasse) from which at least a portion of the sugar has been removed. In some embodiments, a sugar production system that is utilized in an integrated biorefinery system as described herein is a sugar mill. Sugar-containing liquid streams (e.g., cane juice and/or molasses) and/or residual biomass (e.g., bagasse) may be utilized for downstream product production in microbial fermentation systems. For example, microbial (e.g., bacterial; fungal) fermentation processes for production of products of interest may metabolize sugar molecules in liquid sugar-containing streams from the sugar production facility as a source of carbon and/or nutrients (e.g., including but not limited to nitrogen, phosphorous, amino acids, vitamins, trace elements, and/or nucleotides) for production of other products.

In a sugar production facility, such as a sugar mill, sugar molecules that are not bound in polymeric form in the biomass are removed from the biomass, for example, by crushing, milling, pulverizing, washing, rinsing, diffusion processing, heating, adjustment of pH, etc. In one embodiment, the sugar production facility contains a diffuser type sugar mill. In another embodiment, the sugar production facility contains a crush type sugar mill.

In some embodiments, at least a portion of sugar molecules (e.g., hexose (C6) sugar molecules (e.g., sucrose)) that are not bound in polymeric form in the biomass are removed from the biomass in the sugar production facility and converted to one or more liquid sugar-containing streams, such as cane juice and/or molasses (e.g., liquid hexose sugar-containing streams). Such sugar molecules (e.g., hexose sugar molecules) that do not require depolymerization for removal from the biomass are sometimes referred to as free sugar (e.g., free hexose).

In integrated biorefinery systems described herein, a liquid sugar-containing extract from a sugar production facility is provided to a bioproduct production facility and included in a growth medium to support growth and bioproduct production by a microorganism. The microorganism ferments the sugar molecules to produce one or more bioproduct(s) of interest. In some embodiments, the microorganism produces one or more solvents. For example, the microorganism may produce butanol, and optionally other solvents such as acetone. In some embodiments, the microorganism produces butanol. In some embodiments, the microorganism produces acetone. In some embodiments, the microorganism produces butanol and acetone.

In integrated biorefinery systems described herein, residual solid biomass material that remains after sugar processing (e.g., bagasse) and/or biomass material that was removed before sugar processing (e.g., cane straw) may be processed to release additional sugar molecules for use in production of bioproduct(s). In some embodiments, residual sugar molecules (e.g., sucrose) that are not part of the polymeric (e.g., cellulose, hemicellulose) carbohydrate structure of the biomass and that were not extracted in the sugar production facility are mechanically removed from the residual biomass material (e.g., bagasse), for example, by washing. Water and/or vinasse (e.g., bioproduct vinasse (e.g., butanol vinasse) prepared by removal of bioproduct(s) of interest (e.g. butanol) from fermentation broth in a bioproduct fermentation process; and/or ethanol vinasse prepared by removal of ethanol from fermentation broth in an ethanol fermentation process) may be used for such a washing process.

In some embodiments, in addition to or in the absence of washing to remove residual sugar from the biomass, polymeric carbohydrate structural elements of the biomass (such as hemicellulose and/or cellulose) may be hydrolyzed to release soluble sugar molecules. In some embodiments, an acid hydrolysis process is used. For example, a mineral acid (e.g., nitric acid) may be used to hydrolyze residual biomass material from the sugar production facility (e.g., bagasse; and/or cane straw). In one embodiment, a low severity acid hydrolysis process is employed to extract sugar molecules (e.g., C5 sugar molecules) from hemicellulose. Nonlimiting examples of such a process may be found in PCT/US12/070744, which is incorporated by reference herein. In other embodiments, an enzymatic hydrolysis process is used. In one embodiment, sugar molecules are extracted from hemicellulose by acid hydrolysis, then sugar molecules are extracted from residual unhydrolyzed material (e.g., cellulose) with one or more enzyme(s), e.g., cellulase(s). In some embodiments, vinasse is used in a washing process to recover additional sugar from solid residue of hydrolyzed biomass material (e.g., residual solid material after acid hydrolysis). In one embodiment, a countercurrent cascade procedure is used for washing of the biomass material (e.g., hydrolyzed bagasse) with vinasse (e.g., butanol vinasse, ethanol vinasse, or a combination thereof) to remove residual sugar, which may be added to sugar molecules in the liquid hydrolysate, for inclusion in a microbial fermentation medium, or may be added to fermentation medium separately from the liquid hydrolysate

An integrated biorefinery system as disclosed herein includes a bioproduct production facility. A bioproduct production facility includes one or more microorganism(s) that produce one or more product(s) of interest via microbial fermentation of sugar molecules from liquid sugar-containing streams (e.g., cane juice; molasses) from the sugar production facility and/or sugar molecules extracted from residual biomass material from the sugar production facility (e.g., bagasse; cane straw), by washing of residual sugar from bagasse after sugar processing and/or by hydrolysis of one or more polymeric carbohydrate components (e.g., hemicellulose; cellulose) of the biomass (e.g., bagasse and/or cane straw). The bioproduct production facility may include one or more bioreactor(s) for microbial fermentation to produce product(s) of interest. A bioreactor may contain one or more bioproduct-producing microorganism(s) in a growth medium that contains soluble sugar molecules and other nutrients for microbial growth and bioproduct production. In some embodiments, the microorganism(s) are immobilized on a solid support in the bioreactor.

In some embodiments, the bioproduct production facility contains a biomass pretreatment unit, for example, for pretreatment (e.g., hydrolysis) of biomass (e.g., bagasse; cane straw) from the sugar production facility. In some embodiments, the bioproduct production facility contains one or more of a biomass pretreatment unit, a solid-liquid separation unit for separating liquid biomass hydrolysate from residual solid material remaining after hydrolysis, a media preparation unit for preparing microbial growth medium, and product recovery and/or separation units for recovering bioproduct(s) from the fermentation medium, in addition to the bioreactor for bioproduct production. All or a portion of these units may operate continuously and in fluid communication with one another.

In some embodiments, the sugar production facility and the bioproduct production facility operate continuously and in fluid communication with one another. For example, the sugar production facility may continuously produce liquid sugar-containing streams such as cane juice and/or molasses, and may continuously supply these streams to the bioproduct production facility. The sugar-containing streams from the sugar processing facility may be continuously processed and continuously fed to a microbial fermentation medium (e.g., continuously fed into a bioreactor in which microbial bioproduct production proceeds in a continuous process). In some embodiments, effluent may be continuously withdrawn from the bioreactor, bioproduct(s) may be continuously removed from the effluent, thereby producing vinasse, and vinasse may be continuously recycled to the fermentation medium and/or to an upstream process such as biomass hydrolysis.

In some embodiments, sugar-containing liquid from sugar cane processing in the sugar production facility (e.g., cane juice and/or molasses) is included in the growth medium of a microorganism in a bioproduct production facility for production of one or more bioproduct(s) of interest. In other embodiments, sugar molecules that are extracted from residual biomass material (e.g., bagasse) that remains after processing in the sugar production facility, for example, sugar molecules that are extracted by washing of the residual biomass material (e.g., with water and/or vinasse) and/or a hydrolysate that contains sugar molecules extracted by hydrolysis (e.g., acid and/or enzymatic hydrolysis) of residual biomass material such as, for example, bagasse and/or cane straw are included in the growth medium of a microorganism in a bioproduct production facility for production of one or more bioproduct(s) of interest. In other embodiments, both sugar-containing liquid from sugar cane processing in the sugar production facility (e.g., cane juice and/or molasses) and sugar molecules that are extracted from residual biomass material (e.g., bagasse) that remains after processing in the sugar production facility, for example, sugar molecules that are extracted by washing of the residual biomass material (e.g., with water and/or vinasse) and/or a hydrolysate that contains sugar molecules extracted by hydrolysis (e.g., acid and/or enzymatic hydrolysis) of residual biomass material such as, for example, bagasse and/or cane straw are included and co-utilized in the growth medium of a microorganism in a bioproduct production facility for production of one or more bioproduct(s) of interest. In some embodiments, nonlimiting exemplary percent ratios of sugar molecules from sugar-containing liquid from sugar cane processing (e.g., cane juice and/or molasses):hydrolysate (e.g., bagasse and/or cane straw hydrolysate):residual sugar (e.g., free sucrose) washed from bagasse may be about 49:49:2 or about 50:50:0, to about 0:90:10, or about 0:95:5.

In some embodiments, the bioproduct(s) of interest include one or more solvents. In some embodiments, butanol is produced in the bioproduct production facility. In some embodiments, acetone is produced in the bioproduct production facility. In some embodiments, butanol and acetone are produced in the bioproduct production facility.

An integrated biorefinery system as disclosed herein may optionally further include an ethanol production facility. An ethanol production facility includes one or more microorganism(s) (e.g., yeast) that produce ethanol via microbial fermentation of sugar molecules from liquid sugar-containing streams (e.g., cane juice; molasses) from the sugar production facility. The ethanol production facility may include one or more bioreactor(s) for microbial fermentation to produce ethanol. A bioreactor may contain the ethanol-producing microorganism(s) in a growth medium that contains soluble sugar molecules from the sugar production facility and other nutrients for microbial growth and ethanol production. Vinasse and/or killed yeast cells from the ethanol production facility may be supplied to the bioproduct production facility, for example, for use in the microbial growth medium for bioproduct production. In some embodiments, the sugar production facility, the ethanol production facility, and the bioproduct production facility operate continuously and in fluid communication with one another.

In some embodiments, vinasse that is prepared by removal of bioproducts from the fermentation medium in the bioproduct production facility and/or by removal of ethanol from an upstream ethanol production facility is introduced into the fermentation medium in the bioproduct production facility as a source of liquid and/or nutrients for the fermentation. In one embodiment, the vinasse is butanol vinasse. In another embodiment, the vinasse is ethanol vinasse. In another embodiment, the vinasse is a combination of butanol vinasse and ethanol vinasse.

In some embodiments, the fermentation medium in the bioproduct production facility contains about 5% to about 85% vinasse (v/v) (e.g., butanol vinasse and/or ethanol vinasse). In some embodiments, the fermentation medium contains about 5%, about 10%, about 15%, about 20%, about 25%, about 30%, about 35%, about 40%, about 45%, about 50%, about 55%, about 60%, about 65%, about 70%, about 75%, about 80%, or about 85% vinasse (e.g., butanol vinasse and/or ethanol vinasse).

In some embodiments, the fermentation medium in the bioproduct production facility contains about 40% to about 90% (v/v) biomass hydrolysate (e.g., hydrolysate of bagasse and/or cane straw), about 0.1% to about 20% (v/v) cane juice and/or molasses, and about 9.9% to about 60% (v/v) vinasse (e.g., butanol vinasse and/or ethanol vinasse). In various embodiments, the fermentation medium may contain any of about 40%, 45%, 50%, 55%, 60%, 65%, 70%, 75%, 80%, 85%, or 90% hydrolysate of bagasse, hydrolysate of cane straw, or a combination of bagasse and cane straw hydrolysate; any of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% cane juice, molasses, or a combination of cane juice and molasses; and any of about 9.9%, 10%, 15%, 20%, 25%, 30%, 35%, 40%, 45%, 50%, 55%, or 60% vinasse (e.g., butanol vinasse, ethanol vinasse, or a combination of butanol vinasse and ethanol vinasse). The fermentation medium may contain biomass hydrolysate that includes bagasse hydrolysate and cane straw hydrolysate in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0. The fermentation medium may contain cane juice and molasses in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0. The fermentation medium may contain butanol vinasse and ethanol vinasse in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0.

In some embodiments, spent microorganisms (e.g., killed yeast cells) that are used to produce ethanol in the ethanol production facility are used to provide nutrients to the bioproduct production process. In one embodiment, the microorganism in the ethanol production process is yeast. The ethanol-producing microorganism (e.g., yeast) may be added during hydrolysis of the biomass material (e.g., bagasse) and included in the hydrolysate that is provided to the fermentation medium in the bioproduct production facility.

In some embodiments, gases that are produced as byproducts in the bioproduct production facility (e.g., CO₂ and/or H₂) may be recovered. Inn one embodiment, gas (e.g., CO₂ and/or H₂) that is produced during bioproduct fermentation may be recycled and used in place of nitrogen for deaeration of fermentation medium. In other embodiments, gas generated from the fermentation may be recovered and diverted for other purposes. In some embodiments, gas that is generated in bioproduct fermentation may be passed through a scrubber to remove trace solvents, and carbon dioxide may be recovered using well-known processes in the art. The recovered carbon dioxide may then be compressed, purified, liquefied by cooling, and sold as bulk gas or converted to dry ice. Hydrogen, which may be included in the exit gas from the carbon dioxide recovery, may be recovered and used for other purposes.

In some embodiments, spent microorganisms that are used to produce the bioproduct(s) of interest in the bioproduct production facility are processed (e.g., by agglomeration) and incorporated into downstream products, for example, animal feed, soil amendment, and/or roadway amendment. In some embodiments, cell mass is recovered from the stillage at the bottom of a bioproduct distillation column. The stillage may be evaporated to form a thick concentrate. In one embodiment, the thick concentrate is spray dried. The stillage may alternatively be incinerated. Stillage concentrate (e.g., spray dried concentrate) or incinerated stillage may be incorporated, for example, into animal feed, and/or used as a soil amendment and/or roadway amendment.

In some embodiments, butanol is produced in the bioproduct production facility. The butanol may be recovered from the fermentation broth and purified, and may optionally be converted via downstream processes into other molecules.

### Feedstock

A feedstock is a substance that provides the base material from which sugar molecules are generated for inclusion in a microbial growth medium, to support the growth of the microorganism. Feedstock used in the methods described herein contains cellulose and hemicellulose. For example, the feedstock may be lignocellulosic biomass, which contains cellulose, hemicellulose, and lignin. Lignocellulose contains a mixture of carbohydrate polymers and non-carbohydrate compounds. The carbohydrate polymers contain cellulose and hemicellulose, and the non-carbohydrate portion contains lignin. The non-carbohydrate portion may also contain ash, extractives, and/or other components such as proteins. The specific amounts of cellulose, hemicellulose, and lignin depend on the source of the biomass.

Cellulose, which is a β-glucan built up of D-glucose units linked by β(1,4)-glycosidic bonds, is the main structural component of plant cell walls and typically constitutes about 35-60% by weight (% w/w) of lignocellulosic materials.

Hemicellulose refers to non-cellulosic polysaccharides associated with cellulose in plant tissues. Hemicellulose frequently constitutes about 20-35% w/w of lignocellulosic materials, and the majority of hemicelluloses consist of polymers based on pentose (five-carbon) sugar units, such as D-xylose and D-arabinose units, hexose (six-carbon) sugar units, such as D-glucose and D-mannose units, and uronic acids such as D-glucuronic acid.

Lignin, which is a complex, cross-linked polymer based on variously substituted p-hydroxyphenylpropane units, typically constitutes about 10-30% w/w of lignocellulosic materials.

An exemplary feedstock in the systems and methods described herein is bagasse, e.g., sugarcane or sorghum bagasse. Bagasse is the residual fiber generated as part of the sugar extraction process from sugarcane or sorghum, for example, in a sugar processing facility such as a sugar mill. Bagasse contains hemicellulose, cellulose, lignin, and some residual sugars (e.g., residual non-polymeric sugar such as free hexose sugar. In some embodiments, bagasse may contain residual free sugar (e.g., sucrose) that was not removed during sugar processing. In some embodiments, residual sucrose may be removed from bagasse, for example, by mechanical methods such as washing, and hydrolyzed along with sugar molecules from hemicellulose and/or cellulose. For example, during acid hydrolysis, the sucrose may be hydrolyzed to glucose and fructose, which will be included with the soluble sugar molecules in the hydrolysate, in addition to sugar molecules extracted from hemicellulose and cellulose carbohydrate polymers.

Another exemplary feedstock for use in the systems and methods described herein is “cane straw.” Cane straw includes biomass material that is removed prior to processing in a sugar production facility. For example, cane straw may include leaves and/or tops of sugar cane that are cut off and removed prior to processing of sugar cane in a sugar processing facility.

In some embodiments, an amount of feedstock that is used in a method disclosed herein is calculated as dry weight of biomass.

### Pretreatment of Feedstock

Feedstocks such as those described herein (e.g., bagasse; cane straw) can be pretreated using a variety of methods and systems prior to bioconversion to one or more bioproduct(s) of interest. Preparation of the feedstock can include chemical or physical modification of the feedstock. For example, the feedstock can be shredded, sliced, chipped, chopped, heated, burned, dried, separated, extracted, hydrolyzed, milled, and/or degraded. These modifications can be performed by biological, chemical, biochemical and/or mechanical processes.

Typically, a feedstock contains sugar molecules in a polymeric form, and must be hydrolyzed to extract and release soluble monomeric and/or multimeric sugar molecules, which are converted to bioproduct(s), e.g., solvent(s), in a microbial fermentation as described herein. In some embodiments, the sugar molecules are present in the feedstock in cellulose and/or hemicellulose. In one embodiment, the feedstock is lignocellulosic biomass and the sugar molecules are present in the feedstock in cellulose and hemicellulose. Processes may be used to break down cellulose and/or hemicellulose into sugar molecules that may be more easily processed by a microorganism. Processes that may be used include acid hydrolysis, enzymatic hydrolysis, gasification, pyrolysis, and cellulose degradation by a microorganism.

In some embodiments, cellulosic materials may be converted into fermentable sugars by autohydrolysis, e.g., with acetic acid. For example, many lignocellulosic materials contain significant quantities of acetylated hemicellulose. Exposure to high temperature steam may release acetic acid, which may then hydrolyze hemicellulose and/or cellulose to release sugar molecules.

In some embodiments, fermentable sugars may be released from cellulosic materials by enzymatic hydrolysis. For example, cellulose and/or hemicellulose may be treated with hydrolytic enzymes that release mono- and/or disaccharides.

In some embodiments, the feedstock is pretreated with an acid hydrolysis process. Acids that may be used for hydrolysis include, but are not limited to, nitric acid, formic acid, acetic acid, phosphoric acid, hydrochloric acid, and sulfuric acid, or a combination thereof.

Any acid concentration may be used that is suitable for depolymerization of sugar molecules from at least one polymeric component, and that will produce soluble sugar molecules that will support a microbial fermentation process. For example, an acid (e.g., nitric acid) may be used for hydrolysis at a concentration of about 0.5% (w/w) to about 8.5% (w/w), for example, any of about 0.5% to about 1.5%, about 1.5% to about 3.0%, about 3.0% to about 4.5%, about 5.0% to about 6.5%, or about 6.5% to about 8.5%, or any of about 0.5%, 0.6%, 0.7%, 0.8%, 0.9%, 1.0%, 1.5%, 2.0%, 2.5%, 3.0%, 4.0%, 4.5%, 5.0%, 5.5%, 6.0%, 6.5%, 7.0%, 7.5%, 8.0%, or 8.5%. In some embodiments, a polyol (e.g., glycerol) may optionally be included in the hydrolysis mixture at a concentration of about 0.5% (w/w) to about 8.5% (w/w), for example, any of about 0.5% to about 1.5%, about 1.5% to about 3.0%, about 3.0% to about 4.5%, about 5.0% to about 6.5%, or about 6.5% to about 8.5%, or any of about 0.5%, 0.6%, 0.7%, 0.8%, 0.9%, 1.0%, 1.5%, 2.0%, 2.5%, 3.0%, 4.0%, 4.5%, 5.0%, 5.5%, 6.0%, 6.5%, 7.0%, 7.5%, 8.0%, or 8.5%.

In one embodiment, biomass (e.g., bagasse and/or cane straw) is hydrolyzed with acid, e.g., nitric acid, in a low severity process that hydrolyzes primarily hemicellulose, producing a liquid hydrolysate that includes soluble C5 sugar molecules and residual solid material that includes cellulose and lignin. Nonlimiting examples of low severity acid hydrolysis processes which may be used for acid hydrolysis of biomass (e.g., bagasse and/or cane straw) in the methods and systems disclosed herein is described in PCT/US12/070744, which is incorporated by reference herein.

In some embodiments, bagasse and cane straw are hydrolyzed separately and then optionally combined for inclusion in a fermentation medium. In other embodiments, bagasse and cane straw are combined in the same hydrolysis mixture. Bagasse and cane straw may be combined, as biomass for production of a hydrolysate, in any weight ratio, such as for example, about 1 unit cane straw: about 2 units bagasse. In some embodiments, bagasse and cane straw are combined prior to or during hydrolysis in weight percent ratios of any of about 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, or 95:5. In some embodiments, bagasse and cane straw and hydrolyzed separately and the hydrolysates are combined prior to or after addition to fermentation medium in any ratio such as about 1 unit cane straw hydrolysate:about 2 units bagasse hydrolysate. In some embodiments, bagasse and cane straw are hydrolyzed separately and the hydrolysates are combined prior to or after addition to fermentation medium in percent ratios of any of about 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, or 95:5.

In one embodiment, biomass (e.g., bagasse and/or cane straw) is hydrolyzed in a method that includes (a) contacting the biomass with about 0.5% nitric acid (w/w), about 0.5% glycerol (w/w), and water, thereby producing acid impregnated biomass; and (b) feeding the acid impregnated biomass into a digestor through a pressure changing device, wherein the acid impregnated biomass is heated in the digestor at about 140° C. to about 145° C. for about 40 minutes to about 45 minutes, thereby producing a composition that includes a liquid hydrolysate and residual solids, wherein the liquid hydrolysate includes soluble sugar molecules.

In one embodiment, biomass (e.g., bagasse and/or cane straw) is hydrolyzed in a method that includes (a) contacting biomass with acid, e.g., nitric acid, at a concentration of about 0.7% (w/w) to about 1.5% (w/w), thereby producing acid impregnated first biomass; and (b) feeding the acid impregnated first biomass into a digestor through a pressure changing device, wherein the acid impregnated biomass is heated in the digestor at a temperature about 100° C. to about 160° C., and wherein the residence time in the digestor is about 10 minutes to about 120 minutes, thereby producing a composition that comprises soluble sugar molecules.

In one embodiment, the biomass (e.g., bagasse and/or cane straw) is pretreated in a method that includes deconstructing and extracting sugar molecules from the biomass, including: (a) mechanically disintegrating (e.g., reducing the particle size of) the biomass in the presence of water and under a first pressure, thereby producing liquid and/or vapor and solid disintegrated (e.g., size reduced) biomass; (b) separating the liquid and/or vapor from the solid disintegrated biomass (e.g., biomass with reduced particle size), wherein step (b) may be performed after or in conjunction with step (a); (c) contacting the disintegrated biomass with acid, i.e., acid catalyst, e.g., nitric acid, at a concentration sufficient to hydrolyze and/or depolymerize a polymeric carbohydrate component of the biomass, thereby producing acid impregnated disintegrated biomass; and (d) feeding the acid impregnated material into a digestor through a pressure changing device, wherein the acid impregnated material is heated under a second pressure in the digestor at a temperature and for an amount of time sufficient to permit the hydrolysis and/or depolymerization reaction to occur, thereby producing a composition that contains a liquid hydrolysate and residual solids. In some embodiments, the resulting composition or the liquid fraction thereof may be used in a fermentation process, e.g., added to a growth medium for a microbial culture, with or without separation of the liquid hydrolysate from residual solids. Optionally, the method includes (e) separating solids from liquids to produce a liquid hydrolysate and residual solids, wherein the liquid hydrolysate contains soluble hemicellulose sugar molecules and the residual solids contain cellulosic fiber, e.g., partially hydrolyzed cellulosic fiber. In some embodiments, the liquid fraction may be used, e.g., to provide a carbon source, for a microbial fermentation process. In some embodiments, the liquid fraction may be recycled and used for hydrolysis of further biomass and extraction of additional sugar molecules, thereby reducing the amount of acid required for the overall process. In alternate embodiments, liquids and solids are not separated. Optionally, the resulting composition containing liquid hydrolysate and residual solids may be used, for example, in a downstream fermentation process, without separation of liquids from solids.

In some embodiments, step (c) includes contacting the disintegrated biomass with one or more polyol (e.g., glycerol, 1,3-propanediol) and with acid, e.g., nitric acid, at a concentration sufficient to hydrolyze and/or depolymerize a polymeric carbohydrate component of the biomass, thereby producing acid impregnated disintegrated biomass. The polyol (e.g., glycerol, 1,3-propanediol) may be added separately from acid (e.g., prior to or after acid) or simultaneously with the acid. In some embodiments, glycerol is added at a concentration of about 0.3% (w/w) to about 1.2% (w/w). In one embodiment, biomass is contacted with glycerol (for example, about 0.3% (w/w) glycerol to about 1.2% glycerol (w/w), followed by acid (e.g., about 0.3% (w/w) to about 1.2% (w/w) nitric acid). For example, biomass may be contacted with 0.5% (w/w) acid (e.g., nitric acid) and 0.5% (w/w) glycerol, at a temperature of about 140° C. to about 145° C. for about 40 minutes to about 45 minutes. In one embodiment, the glycerol is added from a raw or crude (unpurified) glycerol composition, for example, glycerol that is a byproduct from biodiesel production. For example, the crude glycerol stream may contain about 60% to about 80% glycerol (w/w).

In some embodiments, the acid, for example, nitric acid, is at a concentration of about 0.7% (w/w) to about 1.5% (w/w), the digestor is operated at a temperature of about 100° C. to about 140° C., about 145° C., about 150° C., about 155° C., or about 160° C. (e.g., about 100° C. to about 160° C.), corresponding to a second pressure of about 0 psig to about 38 psig (about 100° C. to about 140° C.) or about 0 psig to about 75 psig (about 100° C. to about 160° C.), and the residence time in the digestor is about 10 minutes to about 120 minutes. Alternatively, the acid, for example, nitric acid, is at a concentration of about 0.8% to about 1.2% (w/w), the digestor is operated at a temperature of about 110° C. to about 140° C., corresponding to a second pressure of about 6 psig to about 38 psig, and the residence time in the digestor is about 20 minutes to about 90 minutes. Alternatively, the acid, for example, nitric acid, is at a concentration of about 0.9% to about 1.2% (w/w), the digestor is operated at a temperature of about 120° C. to about 130° C., corresponding to a second pressure of about 14 psig to about 24 psig, and the residence time in the digestor is about 45 minutes to about 60 minutes.

In some embodiments of any of the above methods, the liquid and/or vapor that is separated from solid disintegrated biomass in step (b) contains extractives.

In some embodiments of any of the above methods, mechanical disintegrating, e.g., particle size reduction, is performed in a thermo-mechanical device. The thermo-mechanical device may be selected from, for example, a modular screw device, an oil press, a disc refiner, and a screw press. Mechanical disintegration, e.g., particle size reduction, may be performed at a pressure and residence time sufficient to shear apart the biomass to make it accessible for acid-catalyzed depolymerization of carbohydrate polymers. In some embodiments, the first pressure is about 5 to about 50 psig and the residence time is about 5 psig to about 60 seconds. For example, mechanical disintegration may be performed at a first temperature of about 70° C. to about 100° C., e.g., about 70° C., about 75° C., about 80° C., about 85° C., about 90° C., about 95° C., or about 100° C. In one embodiment, the first temperature is about 85° C. In an embodiment in which the biomass is bagasse and/or cane straw, e.g., sugarcane bagasse and/or cane straw, mechanical disintegration may serve to remove some extractives and, since the cane juice is acidic, this process may also initiate acid/solid mixing, facilitating acid hydrolysis.

In some embodiments, the digestor is operated under a second temperature of about 100° C. to about 140° C., about 145° C., about 150° C., about 155° C., or about 160° C. (e.g., about 100° C. to about 160° C.), corresponding to a second pressure of about 0 psig to about 38 psig. In some embodiments, the second pressure is higher than the first pressure.

In some embodiments of any of the above methods, the biomass is contacted with steam or other liquids prior to mechanical disintegration, e.g., particle size reduction, which may increase the amount of extractives removed and the degree of disintegration.

In some embodiments, mechanical disintegration of the biomass and associated liquid/solid separation are performed before the acid hydrolysis method, for example, at a separate location, and/or at an earlier time frame prior to contacting the biomass with acid.

In another embodiment, the biomass (e.g., bagasse and/or cane straw) is pretreated in a method that includes: (a) contacting the biomass with acid, i.e., acid catalyst, e.g., nitric acid, at a concentration sufficient to hydrolyze and/or depolymerize a polymeric carbohydrate component of the biomass, thereby producing acid impregnated biomass; and (b) feeding the acid impregnated biomass into a digestor through a pressure changing device, wherein the acid impregnated biomass is heated under pressure in said digestor at a temperature and for an amount of time sufficient to permit the hydrolysis and/or depolymerization reaction to occur. In some embodiments, the resulting composition or the liquid fraction thereof may be used in a fermentation process, e.g., added to a growth medium for a microbial culture, with or without separation of the liquid hydrolysate from residual solids. Optionally, the method includes (c) separating solids from liquids to produce a liquid hydrolysate and residual solids, wherein the liquid hydrolysate contains hemicellulose sugar molecules and the residual solids contain cellulosic fiber, e.g., partially hydrolyzed cellulosic fiber. In some embodiments, the liquid fraction may be used, e.g., to provide a carbon source, for a microbial fermentation process. In some embodiments, the liquid fraction may be recycled and used for hydrolysis of further biomass and extraction of additional sugar molecules, thereby reducing the amount of acid required for the overall process. In alternate embodiments, liquids and solids are not separated. Optionally, the resulting composition containing liquid hydrolysate and residual solids may be used, for example, in a downstream fermentation process, without separation of liquids from solids.

In some embodiments, step (a) includes contacting the biomass with one or more polyol (e.g., glycerol, 1,3-propanediol) and with acid at a concentration sufficient to hydrolyze and/or depolymerize a polymeric carbohydrate component of the biomass, thereby producing acid impregnated disintegrated biomass. The polyol (e.g., glycerol, 1,3-propanediol) may be added separately from acid (e.g., prior to or after acid) or simultaneously with the acid. In some embodiments, glycerol is added at a concentration of about 0.3% (w/w) to about 1.2% (w/w). In one embodiment, biomass is contacted with glycerol (for example, about 0.3% glycerol to about 1.2% glycerol (w/w)), followed by acid (e.g., about 0.3% to about 1.2% (w/w) nitric acid). For example, biomass may be contacted with 0.5% (w/w) acid (e.g., nitric acid) and 0.5% (w/w) glycerol, at a temperature of about 140° C. to about 145° C. for about 40 minutes to about 45 minutes. In one embodiment, the glycerol is added from a raw or crude (unpurified) glycerol composition, for example, glycerol that is a byproduct from biodiesel production. For example, the crude glycerol stream may contain about 60% to about 80% glycerol (w/w).

In some embodiments, the acid, e.g., nitric acid, concentration in step (a) is about 0.7% (w/w) to about 1.5% (w/w), 0.8% (w/w) to about 1.2% (w/w), or about 0.9% (w/w) to about 1.2% (w/w). In some embodiments, the residence time in step (b) is about 10 minutes to about 120 minutes, about 20 minutes to about 90 minutes, or about 45 minutes to about 60 minutes. In some embodiments, the temperature in step (b) is about 100° C. to about 140° C., about 145° C., about 150° C., about 155° C., or about 160° C. (e.g., about 100° C. to about 160° C.), about 110° C. to about 140° C., or about 120° C. to about 130° C. In some embodiments, the acid concentration in step (a) is about 0.7% (w/w) to about 1.5% (w/w), the residence time in step (b) is about 10 minutes to about 120 minutes, and the temperature in step (b) is about 100° C. to about 140° C., corresponding to a pressure of about 0 psig to about 38 psig (about 100° C. to about 140° C.) or about 0 psig to about 75 psig (about 100° C. to about 160° C.). In other embodiments, the acid concentration in step (a) is about 0.8% (w/w) to about 1.2% (w/w), the residence time in step (b) is about 20 minutes to about 90 minutes, and the temperature in step (b) is about 110° C. to about 140° C., corresponding to a pressure of about 6 psig to about 38 psig. In further embodiments, the acid concentration in step (a) is about 0.9% (w/w) to about 1.5% (w/w), the residence time in step (b) is about 45 minutes to about 60 minutes, and the temperature in step (b) is about 120° C. to about 130° C., corresponding to a pressure of about 14 psig to about 24 psig. In some embodiments, the biomass is contacted with steam prior to acid impregnation, which may aid with disintegration of the biomass and extractives removal.

In another embodiment, the biomass (e.g., bagasse and/or cane straw) is pretreated in a method that includes: (a) contacting biomass with acid, glycerol, and water, thereby producing acid impregnated biomass, wherein acid, e.g., nitric acid, is included at a concentration that is sufficient to hydrolyze and/or depolymerize a polymeric carbohydrate component of the biomass; and (b) feeding the acid impregnated material into a digestor through a pressure changing device, wherein the acid impregnated biomass is heated at a temperature and for a time that is sufficient to produce a composition that comprises a liquid hydrolysate and residual solids, wherein the liquid hydrolysate comprises soluble sugar molecules. In one embodiment, the acid is nitric acid, present at a concentration of about 0.5% (w/w), glycerol is present at a concentration of about 0.5% (w/w), and the acid impregnated material is heated in the digestor at about 140° to about 145° C. for about 40 minutes to about 45 minutes. In one embodiment, the glycerol is contained within and added in a crude glycerol composition that includes about 60% to about 80% glycerol by weight in an amount to provide about 0.5% (w/w) glycerol molecules in the acid hydrolysis mixture that includes nitric acid, glycerol, and water.

In another embodiment the biomass (e.g., bagasse and/or cane straw) is pretreated in a method that includes: (a) contacting biomass with about 0.5% nitric acid (w/w), about 0.5% glycerol (w/w), and water, thereby producing acid impregnated biomass; and (b) feeding the acid impregnated biomass into a digestor through a pressure changing device, wherein the acid impregnated biomass is heated in the digestor at about 140° C. to about 145° C. for about 40 minutes to about 45 minutes, thereby producing a composition that comprises a liquid hydrolysate and residual solids, wherein the liquid hydrolysate comprises soluble sugar molecules. In some embodiments, the glycerol is added in a crude glycerol composition that comprises about 60% to about 80% glycerol by weight. In some embodiments, the biomass is mechanically disintegrated prior to or in conjunction with step (a).

In another embodiment, the biomass (e.g., bagasse and/or cane straw) is pretreated in a method that includes: (a) contacting a first biomass with acid, e.g., nitric acid, at a concentration sufficient to depolymerize a polymeric carbohydrate component from the first biomass, thereby producing acid impregnated first biomass; (b) feeding the acid impregnated first biomass into a digestor through a pressure changing device, wherein the acid impregnated first biomass is heated in the digestor at a temperature and for an amount of time sufficient to permit hydrolysis to occur, thereby producing a composition that comprises a first liquid hydrolysate and first residual solids, wherein the first liquid hydrolysate comprises soluble sugar molecules; (c) separating the first liquid hydrolysate from the first residual solids; (d) contacting a second biomass with the first liquid hydrolysate, thereby producing acid impregnated second biomass; and (e) feeding the acid impregnated second biomass into a digestor through a pressure changing device, wherein the acid impregnated second biomass is heated in the digestor at a temperature and for an amount of time sufficient to permit hydrolysis to occur, thereby producing a composition that comprises a second liquid hydrolysate and second residual solids, wherein the second liquid hydrolysate comprises soluble sugar molecules, and wherein the amount of soluble sugar molecules in the second liquid hydrolysate is greater than the amount of soluble sugar molecules in the first liquid hydrolysate. In some embodiments, the first biomass is mechanically disintegrated prior to or in conjunction with step (a). In one embodiment, the acid concentration in step (a) is about 0.7% (w/w) to about 1.5% (w/w), the digestor in step (b) is operated at a temperature of about 100° C. to about 160° C. with a residence time of about 10 minutes to about 120 minutes, and the digestor in step (e) is operated at a temperature of about 100° C. to about 160° C. with a residence time of about 10 minutes to about 120 minutes.

In another embodiment, the acid concentration in step (a) is about 0.8% (w/w) to about 1.2% (w/w), the digestor in step (b) is operated at a temperature of about 110° C. to about 140° C. with a residence time of about 20 minutes to about 90 minutes, and the digestor in step (e) is operated at a temperature of about 110° C. to about 140° C. with a residence time of about 20 minutes to about 90 minutes.

In a further embodiment, the acid concentration in step (a) is about 0.9% (w/w) to about 1.2% (w/w), the digestor in step (b) is operated at a temperature of about 120° C. to about 130° C. with a residence time of about 45 minutes to about 60 minutes, and the digestor in step (e) is operated at a temperature of about 120° C. to about 130° C. with a residence time of about 45 minutes to about 60 minutes.

In some embodiments, the method includes contacting the first biomass with a polyol, wherein the polyol is added separately from the acid in step (a) or simultaneously with the acid in step (a). In one embodiment, the polyol includes glycerol. In some embodiments, glycerol is added in a crude glycerol composition that includes about 60% to about 80% glycerol by weight. In one embodiment, glycerol is included in step (a) at a concentration of about 0.3% (w/w) to about 1.2% (w/w). In one embodiment, the acid in step (a) is nitric acid at a concentration of about 0.3% (w/w) to about 1.2% (w/w). In one embodiment, step (a) comprises contacting the first biomass with about 0.5% (w/w) nitric acid and about 0.5% (w/w) glycerol, wherein the digestor in step (b) is operated at a temperature of about 140° C. to about 145° C. with a residence time of about 40 minutes to about 45 minutes, and wherein the digestor in step (e) is operated at a temperature of about 140° C. to about 145° C. with a residence time of about 40 minutes to about 45 minutes.

In another embodiment, the biomass (e.g., bagasse and/or cane straw) is pretreated in a method that includes: (a) contacting biomass with acid, e.g., nitric acid, at a concentration of about 0.7% (w/w) to about 1.5% (w/w), thereby producing acid impregnated first biomass; and (b) feeding the acid impregnated first biomass into a digestor through a pressure changing device, wherein the acid impregnated biomass is heated in the digestor at a temperature about 100° C. to about 160° C., and wherein the residence time in the digestor is about 10 minutes to about 120 minutes, thereby producing a composition that comprises soluble sugar molecules. In some embodiments, the composition that comprises soluble sugar molecules comprises a liquid hydrolysate and residual solids, and the method further includes: (c) separating the liquid hydrolysate from the residual solids. In one embodiment, the acid concentration in step (a) is about 0.8% (w/w) to about 1.2% (w/w), wherein the temperature in the digestor in step (b) is about 110° C. to about 140° C., and wherein the residence time in the digestor in step (b) is about 20 minutes to about 90 minutes. In another embodiment, the acid concentration in step (a) is about 0.9% (w/w) to about 1.2% (w/w), wherein the temperature in the digestor in step (b) is about 120° C. to about 130° C., and wherein the residence time in the digestor in step (b) is about 45 minutes to about 60 minutes. In some embodiments, the biomass is mechanically disintegrated prior to or in conjunction with step (a). In some embodiments, the method includes contacting the biomass with a polyol, wherein the polyol is added separately from the acid in step (a) or simultaneously with the acid in step (a). In one embodiment, the polyol includes glycerol. In one embodiment, glycerol is added in a crude glycerol composition that includes about 60% to about 80% glycerol by weight. In one embodiment, the acid concentration in step (a) is about 0.3% (w/w) to about 1.2% (w/w), and wherein glycerol is included in step (a) at a concentration of about 0.3% (w/w) to about 1.2% (w/w).

In some embodiments of any of the above methods, hemicellulose and optionally some cellulose may be depolymerized from the biomass material, and the hydrolysate contains soluble sugar molecules from hemicellulose and optionally some sugar molecules from cellulose.

In some embodiments of any of the above methods, the pressure changing device in the digestor is selected from a plug screw feeder, a rotary valve, a low pressure feeder, or a lockhopper arrangement. In some embodiments, heating of the acid impregnated biomass (e.g., acid impregnated disintegrated biomass) in the digestor is with direct or indirect steam. In some embodiments of any of the above methods, the digestor is operated under pressure. In one embodiment, the digestor is a continuous feed, pressure rated, screw conveyor vessel. In some embodiments, the material that is fed to the digestor comprises a liquid to solid ratio of about 1:1 to about 20:1. In some embodiments in which a recirculating reactor is used, the liquid to solid ratio may be about 9:1. In embodiments in which a plug flow reactor is used, the liquid to solid ratio may be about 2:1 to about 3:1, or about 2:1 to about 4:1.

In some embodiments of any of the above methods, the liquid hydrolysate contains about 10 g/l to about 150 g/l, about 40 g/l to about 100 g/l, about 60 g/l to about 90 g/l, or about 65 g/l to about 80 g/l soluble sugar molecules. In some embodiments, the soluble sugar molecules include xylose. In some embodiments, the soluble sugar molecules include mannose, xylose, glucose, arabinose, and galactose.

Liquid hydrolysate that includes soluble sugar molecules (e.g., from depolymerization of hemicellulose) may optionally be separated from residual solids prior to inclusion in a fermentation medium.

The residual solids may be further hydrolyzed to release further soluble sugar molecules (e.g., from depolymerization of cellulose) and/or may be used as a fuel source, e.g., as fuel for a boiler in the integrated biorefinery system and/or for electricity generation.

In one embodiment, further hydrolysis of residual solids, e.g., containing cellulose and lignin, may be hydrolyzed with one or more enzyme that is capable of depolymerizing cellulose, e.g., hydrolysis of 1,4-beta-D-glycosidic linkages in cellulose. For example, enzymatic hydrolysis may be performed with one or more cellulase enzyme(s). Nonlimiting examples of cellulase enzymes include endocellulases, exocellulases, cellobiases, oxidative cellulases, and cellulose phosphorylases. Optionally, liquid hydrolysate that includes soluble sugar molecules (e.g., from depolymerization of cellulose) may optionally be separated from residual solids prior to inclusion in a fermentation medium, and optionally, the solids (e.g., lignin) may be used as a fuel source, e.g., as fuel for a boiler in the integrated biorefinery system and/or for electricity generation. In another embodiment, further hydrolysis of residual solids, e.g., containing cellulose and lignin, may be hydrolyzed with acid, e.g., acid, under conditions suitable for depolymerization of cellulose. For example, hydrolysis is performed with an acid, e.g., nitric acid, concentration of about 0.05% to about 0.1%, about 0.1% to about 0.5%, about 0.5% to about 1%, about 1% to about 4%, about 1.3% to about 3.5%, or about 1.3% (w/w of dry feedstock) at a temperature of about 210° to about 230° C., and at the saturation pressure for steam at the reactor temperature. Optionally, liquid hydrolysate that includes soluble sugar molecules (e.g., from depolymerization of cellulose) may optionally be separated from residual solids prior to inclusion in a fermentation medium, and optionally, the solids (e.g., lignin) may be used as a fuel source, e.g., as fuel for a boiler in the integrated biorefinery system and/or for electricity generation.

In any of the biomass hydrolysis methods described herein, solids may optionally be separated from liquids to produce a hydrolysate and residual solids in a screw press, belt filter press, centrifuge, settling tank, vacuum filter, sieve screen, or rotary drum dryer.

In some embodiments, the hydrolysate from the second hydrolysis (e.g., hydrolysate containing depolymerized cellulose) is combined with the hydrolysate from the first hydrolysis (e.g., hydrolysate containing depolymerized hemicellulose) and the combined hydrolysates are included in a fermentation to produce one or more bioproduct(s) of interest in a bioproduct production facility as disclosed herein. In other embodiments, the hydrolysate from the second hydrolysis (e.g., hydrolysate containing depolymerized cellulose) and the hydrolysate from the first hydrolysis (e.g., hydrolysate containing depolymerized hemicellulose) are separately fed to separate bioreactors for production of one or more bioproduct(s) of interest. The separate bioreactors may contain the same or different microorganisms and may produce the same or different bioproduct(s). In one embodiments, the first hydrolysate is fed to a bioreactor that contains a microorganism that is optimized for growth in the presence of this hydrolysate (e.g., hydrolysate containing C5 and C6 sugar molecules), and the second hydrolysate is fed to a bioreactor than contains a microorganism that is optimized for growth in the presence of this second hydrolysate (e.g., hydrolysate containing C6 sugar molecules).

### Conditioning of Hydrolyzed Feedstock

In some embodiments, hydrolysate, e.g., hydrolyzed bagasse and/or cane straw, produced as described herein, is “conditioned” to remove inhibitors of microbial growth and/or bioproduct, production and/or to adjust one or more parameters of the hydrolysate to render it more suitable for addition to a microbial growth medium, for example, adjustment of pH and/or temperature to a physiologically acceptable level for growth of a microorganism when added to microbial growth medium.

In some embodiments of the methods disclosed herein, a biomass hydrolysate (e.g., a hydrolysate of bagasse and/or cane straw) is rendered fermentable, i.e., suitable for microbial fermentation, after raising the pH to a physiologically acceptable level for growth of a particular microbial culture, for example, from the pH of the hydrolysate after acid hydrolysis (e.g., about pH 2.5) to about pH 6 to 7 (e.g., about 6.7). In some embodiments, no further conditioning processes are required, other than the pH adjustment, for the hydrolysate to support microbial growth and/or bioproduct production (i.e., treatment of the hydrolysate to remove inhibitors is not required). Although not wishing to be bound by theory, raising the pH may result in deprotonation of certain organic acid inhibitor compounds, rendering them less inhibitory.

In some embodiments, conditioning processes are included for removal of inhibitors from the hydrolysate. Inhibitors of microbial growth and/or bioproduct production may include, but are not limited to, organic acids, furans, phenols, soluble lignocellulosic materials, extractives, and ketones. Inhibitors present in hydrolysates may include, but are not limited to, 5-hydroxyy-methyl furfural (HMF), furfural, aliphatic acids, levulinic acid, acetic acid, formic acid, phenolic compounds, vanillin, dihydroconiferylalcohol, coniferyl aldehyde, vanillic acid, hydroquinone, catechol, acetoguaiacone, homovanillic acid, 4-hydroxy-benzoic acid, Hibbert's ketones, ammonium nitrate and/or other salts, p-coumaric acid, ferulic acid, vanillic acid, syringaldehyde, sinapyl alcohol, and glucuronic acid.

Nonlimiting examples of conditioning processes for removal of inhibitors include vacuum or thermal evaporation, overliming, precipitation, adsorption, enzymatic conditioning (e.g., peroxidase, laccase), chemical conversion, distillation, evaporation, filtration, and ion exchange, or a combination thereof. In one embodiment, conditioning includes contact of hydrolysate with an ion exchange resin, such as an anion or cation exchange resin. Inhibitors may be retained on the resin. In one embodiment, the ion exchange resin is an anion exchange resin. Ion exchange resins may be regenerated with caustic, some solvents, or other known industrial materials. In other embodiments, inhibitors may be precipitated by a metal salt (for example, a trivalent metal salt, for example, an aluminum or iron salt, such as aluminum sulfate or ferric chloride), calcium based salts (for example, lime) and/or a flocculant such as polyethylene oxide or other low density, high molecular weight polymers.

In one embodiment, hydrolysate is conditioned on ion exchange resin, such as an anion exchange resin, e.g., Duolite A7, at acidic pH, for example, pH about 2.5 to about 5.5, about 3.5 to about 4.5, or about 2.5, 3, 3.5, 4, 4.5, 5, or 5.5.

In one embodiment, hydrolysate is conditioned with calcium oxide or hydroxide (lime). In some embodiments, the lime is added to increase the pH of the hydrolysate to about 12, about 11, or about 10, at a temperature of about 30° C. to about 60° C., for about 30 minutes to about 60 minutes or about 40 minutes to about 50 minutes, or up to about 72 hours. The resulting precipitation process removes calcium salts and condensable lignins and phenolic compounds, rendering the resulting hydrolysate more fermentable.

In one embodiment, hydrolysate is conditioned with a metal salt, for example, a trivalent metal salt, such as an aluminum or iron salt, e.g., aluminum sulfate or ferric chloride. In some embodiments, the metal salt is added at a concentration of about 1 g/L to about 6 g/L, or about 3 g/L to about 5 g/L. In some embodiments, the pH is adjusted with a base to a basic pH, such as about 9.5 to about 11, or about 9.5, 10, 10.5, or 11, for example, with ammonium hydroxide or ammonia gas.

In some embodiments, one or more agent(s) or compound(s) that promotes microbial growth and/or bioproduct production is added to biomass hydrolysate (e.g., a hydrolysate of bagasse and/or cane straw), to a liquid sugar-containing stream from a sugar production facility (e.g., cane juice and/or molasses), and/or to fermentation medium.

In some embodiments, microbial growth and/or bioproduct, e.g., biofuel, titer, yield, and/or productivity is increased when conditioned hydrolyzed feedstock is used, in comparison to identical hydrolyzed feedstock which has not been subjected to the conditioning process.

In some embodiments, a microorganism that is tolerant to inhibitors in hydrolyzed feedstock is used, or the microorganism used for bioproduct production develops increased tolerance to inhibitors over time, e.g., by repeated passaging, rendering the conditioning step unnecessary or uneconomical.

### Methods for Producing a Bioproduct

Methods are provided for producing one or more bioproduct(s) of interest in an integrated biorefinery system as described herein. The systems described herein include a bioproduct production facility. Process streams and/or residual material are provided to the bioproduct production facility from a sugar production facility, e.g., sugar mill, and optionally from an ethanol production facility as disclosed herein.

The methods include culturing a microorganism that produces the bioproduct of interest in a medium that contains a liquid sugar-containing extract (e.g., cane juice and/or molasses) and/or a hydrolysate or conditioned hydrolysate of residual biomass (e.g., bagasse and/or cane straw) from the sugar production facility, as soluble sugar to support microbial growth for production of one or more bioproduct(s) of interest. In some embodiments, the microorganism is cultured in a medium that contains both liquid sugar-containing extract (e.g., cane juice and/or molasses) and a hydrolysate or conditioned hydrolysate of residual biomass (e.g., bagasse and/or cane straw) from the sugar production facility. Optionally, the medium may also contain residual sugar that was mechanically removed (e.g., washed) from the biomass (e.g., bagasse) after sugar processing in the sugar production facility.

In some embodiments, the culture medium may contain vinasse. The vinasse may be recycled medium from the bioproduct production plant, from which bioproduct has been removed, and/or may be medium from another fermentative process, for example, an ethanol production plant (from which ethanol has been removed).

In some embodiments, the fermentation medium in the bioproduct production facility contains about 5% to about 85% vinasse (v/v) (e.g., butanol vinasse and/or ethanol vinasse). In some embodiments, the fermentation medium contains about 5%, about 10%, about 15%, about 20%, about 25%, about 30%, about 35%, about 40%, about 45%, about 50%, about 55%, about 60%, about 65%, about 70%, about 75%, about 80%, or about 85% vinasse (e.g., butanol vinasse and/or ethanol vinasse).

In some embodiments, the fermentation medium in the bioproduct production facility contains about 40% to about 90% (v/v) biomass hydrolysate (e.g., hydrolysate of bagasse and/or cane straw), about 0.1% to about 20% (v/v) cane juice and/or molasses, and about 9.9% to about 60% (v/v) vinasse (e.g., butanol vinasse and/or ethanol vinasse). In various embodiments, the fermentation medium may contain any of about 40%, 45%, 50%, 55%, 60%, 65%, 70%, 75%, 80%, 85%, or 90% hydrolysate of bagasse, hydrolysate of cane straw, or a combination of bagasse and cane straw hydrolysate; any of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% cane juice, molasses, or a combination of cane juice and molasses; and any of about 9.9%, 10%, 15%, 20%, 25%, 30%, 35%, 40%, 45%, 50%, 55%, or 60% vinasse (e.g., butanol vinasse, ethanol vinasse, or a combination of butanol vinasse and ethanol vinasse). The fermentation medium may contain biomass hydrolysate that includes bagasse hydrolysate and cane straw hydrolysate in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0. The fermentation medium may contain cane juice and molasses in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0. The fermentation medium may contain butanol vinasse and ethanol vinasse in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0.

In some embodiments, the culture medium may contain spent yeast cells from an ethanol production process. Yeast cells may be treated with a process that lyses or otherwise kills the cells, prior to inclusion in the bioproduct culture medium. Non-limiting examples of such processes include thermal autolysis or base-catalyzed lysis. Yeast cells may also be hydrolyzed during biomass pretreatment by inclusion in a biomass hydrolysis mixture. In various embodiments, the yeast cells may provide nutrition to the bioproduct-producing microorganism.

In some embodiments, the bioproduct is a biofuel, for example, butanol, acetone, and/or ethanol. In some embodiments, the bioproduct is solvent (e.g., a polar protic or aprotic solvent), biomolecule, organic acid, alcohol, fatty acid, aldehyde, lipid, long chain organic molecule, vitamin, or sugar alcohol. In some embodiments, the bioproduct is a solvent or organic acid.

### Fermentation

The methods for bioproduct production herein include fermentation with a bioproduct-producing microorganism in a bioreactor in a growth medium that contains liquid sugar-containing extract (e.g., cane juice and/or molasses) and/or a hydrolysate or conditioned hydrolysate of residual biomass (e.g., bagasse and/or cane straw) from the sugar production facility.

In some embodiments, the bioproduct production includes fermentation with a bioproduct-producing microorganism in an immobilized cell bioreactor (i.e., a bioreactor containing cells that are immobilized on a support, e.g., a solid support). In some embodiments, an immobilized cell bioreactor provides higher productivity due to the accumulation of increased productive cell mass within the bioreactor compared with a stirred tank (suspended cell) bioreactor. In some embodiments, the microbial cells form a biofilm on the support and/or between support particles in the growth medium.

In other embodiments, for example but not limited to, embodiments in which a hydrolysate composition containing both liquid hydrolysate and solid residues is used, microorganisms may be grown in a non-immobilized system, such as an agitated fermentation reactor, e.g., designed to provide adequate conditions for fermentation, including but not limited to mixing of components, gas removal, temperature control, and/or the ability to add and/or remove material from the reactor. Several fermentation operational moieties exist, including but not limited to batch, fed-batch, and continuous in single or multiple reactor configurations. Exemplar reactor types include but are not limited to agitated tanks, e.g., where agitation is effected by a mechanical impeller, the addition and withdrawal of material, the addition of gas, and/or the recirculation of fermentation gas; corn and/or cane ethanol fermentation tanks; pharmaceutical fermentation vessels; vacuum fermentation systems; air-lift type reactors; fluidized bed reactors; anaerobic digestors; and activated sludge reactors. In some embodiments, an extractive fermentation process is used (e.g., gas stripping, liquid extraction, vacuum fermentation).

In some embodiments, the bioproduct production process herein includes continuous fermentation of a microorganism (continuous addition of conditioned hydrolyzed feedstock and withdrawal of product stream). Continuous fermentation minimizes the unproductive portions of the fermentation cycle, such as lag, growth, and turnaround time, thereby reducing capital cost, and reduces the number of inoculation events, thus minimizing operational costs and risk associated with human and process error.

Fermentation may be aerobic or anaerobic, depending on the requirements of the bioproduct-producing microorganism.

In some embodiments, an immobilized bioproduct-producing Clostridium strain is fermented anaerobically in a continuous or batch process. In some embodiments, the Clostridium strain produces butanol.

One or more bioreactors may be used in the bioproduct production systems and processes described herein. When multiple bioreactors are used they can be arranged in series and/or in parallel. The advantages of multiple bioreactors over one large bioreactor include lower fabrication and installation costs, ease of scale-up production, and greater production flexibility. For example individual bioreactors may be taken off-line for maintenance, cleaning, sterilization, and the like without appreciably impacting the production schedule. In embodiments in which multiple bioreactors are used, the bioreactors may be run under the same or different conditions.

In a parallel bioreactor arrangement, liquid sugar-containing extract and/or hydrolyzed feedstock (e.g., hydrolyzed bagasse and/or cane straw) is fed into multiple bioreactors, and effluent from the bioreactors is removed. The effluent may be combined from multiple bioreactors for recovery of the bioproduct, or the effluent from each bioreactor may be collected separately and used for recovery of the bioproduct.

In a series bioreactor arrangement, liquid sugar-containing extract (e.g., cane juice and/or molasses) and/or hydrolyzed feedstock (e.g., hydrolyzed bagasse and/or cane straw) is fed into the first bioreactor in the series, the effluent from the first bioreactor is fed into a second downstream bioreactor, and the effluent from each bioreactor in the series is fed into the next subsequent bioreactor in the series. The effluent from the final bioreactor in the series is collected and may be used for recovery of the bioproduct.

Each bioreactor in a multiple bioreactor arrangement can have the same species, strain, or mix of species or strains of microorganisms or a different species, strain, or mix of species or strains of microorganisms compared to other bioreactors in the series.

Immobilized cell bioreactors allow higher concentrations of productive cell mass to accumulate and therefore, the bioreactors can be run at high dilution rates, resulting in a significant improvement in volumetric productivity relative to cultures of suspended cells. Since a high density, steady state culture can be maintained through continuous culturing, with the attendant removal of product containing fermentation broth, smaller capacity bioreactors can be used. Bioreactors for the continuous fermentation of C. acetobutylicum are known in the art. (U.S. Pat. Nos. 4,424,275, and 4,568,643.)

Numerous methods of fermentor inoculation are possible including addition of a liquid seed culture to the bottom or the top of the bioreactor and recirculation of the media to encourage growth throughout the bed. Other methods include the addition of a liquid seed culture or impregnated solid support through a port located along the reactor's wall or integrated and loaded with the solid support material. Bioreactor effluent may also be used to inoculate an additional bioreactor and in this case any residual fermentable materials may be converted in the secondary reactor, increasing yield/recovery.

In a similar manner, support material may be added to the reactor through bottom, top, or side loading to replenish support material that becomes degraded or lost from the bioreactor.

### Fermentation Media

Fermentation media for the production of bioproduct(s) may contain liquid sugar-containing extract (e.g., cane juice and/or molasses) and/or sugar molecules extracted from residual biomass material after processing, for example, in a sugar mill as described herein, e.g., bagasse. For example, the fermentation media may contain a hydrolysate or conditioned hydrolysate of residual biomass (e.g., bagasse and/or cane straw) as a source of fermentable carbohydrate molecules. In one embodiment, the fermentation media contains both liquid sugar-containing extract (e.g., cane juice and/or molasses) and sugar molecules extracted from residual bagasse after processing of biomass, e.g., sugar cane, in a sugar production plant. In one embodiment, sugar molecules are extracted from residual biomass (e.g., bagasse and/or cane straw) in a process that includes acid hydrolysis (e.g., nitric acid hydrolysis). In one embodiment, residual free sugar, e.g., sucrose, is removed by a mechanical process such as washing prior to acid hydrolysis and is combined with the hydrolysate in the fermentation media. In some embodiments, the fermentation media contains vinasse (e.g., bioproduct (e.g., butanol) and/or ethanol vinasse). In some embodiments, the fermentation media contains spent yeast cells from an ethanol production facility as described herein. In some embodiments, the fermentation media contains steam condensate from a sugar processing facility as described herein.

As known in the art, in addition to an appropriate carbon source, fermentation media must contain suitable nitrogen source(s), mineral salts, cofactors, buffers, and other components suitable for the growth of the cultures and promotion of the enzymatic pathway necessary for the production of the desired bioproduct. In some embodiments, salts and/or vitamin B12 or precursors thereof are included in the fermentation media. In some cases, hydrolyzed biomass (e.g., bagasse and/or cane straw) may contain some or all of the nutrients required for growth, minimizing or obviating the need for additional supplemental material.

The nitrogen source may be any suitable nitrogen source, including but not limited to, ammonium salts, yeast extract, corn steep liquor (CSL), soy meal and/or other protein sources including, but not limited to, denatured proteins recovered from distillation of fermentation broth or extracts derived from the residual separated microbial cell mass recovered after fermentation. Phosphorus may be present in the medium in the form of phosphate salts, such as sodium, potassium, or ammonium phosphates. Sulfur may be present in the medium in the form of sulfate salts, such as sodium or ammonium sulfates. Additional salts include, but are not limited to, magnesium sulfate, manganese sulfate, iron sulfate, magnesium chloride, calcium chloride, manganese chloride, ferric chloride, ferrous chloride, zinc chloride, cupric chloride, cobalt chloride, and sodium molybdate. The growth medium may also contain vitamins such as thiamine hydrochloride, biotin, and para-aminobenzoic acid (PABA). The growth medium may also contain one or more buffering agent(s) (e.g., MES), one or more reducing agent(s) (e.g., cysteine HCl), and/or sodium lactate, which may serve as a carbon source and pH buffer. Osmoprotectants, such as trehalose, may also be added to the media.

### Microorganisms

A bioproduct production facility as described herein includes one or more microorganism(s) that is (are) capable of producing one or more bioproduct(s) of interest. In embodiments in which two or more microorganisms are used, the microorganisms may be the same or different microbial species and/or different strains of the same species.

In some embodiments, the microorganisms are bacteria or fungi. In some embodiments, the microorganisms are a single species. In some embodiments, the microorganisms are a mixed culture of strains from the same species. In some embodiments, the microorganisms are a mixed culture of different species. In some embodiments, the microorganisms are an environmental isolate or strain derived therefrom.

In some embodiments of the processes and systems described herein, different species or strains, or different combinations of two or more species or strains, are used in different bioreactors with different conditioned hydrolyzed feedstocks as a carbohydrate source.

In some embodiments, a fungal microorganism is used, such as a yeast. Examples of yeasts include, but are not limited to, Saccharomyces cerevisiae, S. bayanus, S. carlsbergensis, S. Monacensis, S. Pastorianus, S. uvarum and Kluyveromyces species. Other examples of anaerobic or aerotolerant fungi include, but are not limited to, the genera Neocallimastix, Caecomyces, Piromyces and other rumen derived anaerobic fungi.

In some embodiments, a bacterial microorganism is used, including Gram-negative and Gram-positive bacteria. Non-limiting examples of Gram-positive bacteria include bacteria found in the genera of Staphylococcus, Streptococcus, Bacillus, Mycobacterium, Enterococcus, Lactobacillus, Leuconostoc, Pediococcus, and Propionibacterium. Non-limiting examples of specific species include Enterococcus faecium and Enterococcus gallinarium. Non-limiting examples of Gram-negative bacteria include bacteria found in the genera Pseudomonas, Zymomonas, Spirochaeta, Methylosinus, Pantoea, Acetobacter, Gluconobacter, Escherichia and Erwinia.

In some embodiments, the microorganisms are from the genera Clostridium, Enterococcus, Klebsiella, Lactobacillus, Enterococcus, Escherichia, Pichia, Pseudomonas, Synechocystis, Saccharomyces, or Bacillus.

In one embodiment, the bacteria are Clostridium species, including but not limited to, Clostridium saccharobutylicum, Clostridium acetobutylicum, Clostridium beijerinckii, Clostridium puniceum, Clostridium saccharoperbutylacetonicum, Clostridium pasteuranium, Clostridium butylicum, Clostridium aurantibutyricum, Clostridium tetanomorphum, Clostridium thermocellum, and Clostridium thermosaccharolyticum, Clostridium cellulolyticum, Clostridium phytofermentans, Clostridium. saccharolyticum, Clsotridium thermobutyricum, and environmental isolates of Clostridium.

Other bacteria contemplated for use in the bioproduct production plant herein include Corynebacteria, such as C. diphtheriae, Pneumococci, such as Diplococcus pneumoniae, Streptococci, such as S. pyogenes and S. salivarus, Staphylococci, such as S. aureus and S. albus, Myoviridae, Siphoviridae, Aerobic Spore-forming Bacilli, Bacilli, such as B. anthracis, B. subtilis, B. megaterium, B. cereus, Butyrivibrio fibrisolvens, Anaerobic Spore-forming Bacilli, Mycobacteria, such as M. tuberculosis hominis, M. bovis, M. avium, M. paratuberculosis, Actinomycetes (fungus-like bacteria), such as, A. israelii, A. bovis, A. naeslundii, Nocardia asteroides, Nocardia brasiliensis, the Spirochetes, Treponema pallidium, Treponema pertenue, Treponema carateum, Borrelia recurrentis, Leptospira icterohemorrhagiae, Leptospira canicola, Spirillum minus, Streptobacillus moniliformis, Trypanosomas, Mycoplasmas, Mycoplasma pneumoniae, Listeria monocytogenes, Erysipelothrix rhusiopathiae, Streptobacillus monilformis, Donvania granulomatis, Bartonella bacilliformis, Rickettsiae, Rickettsia prowazekii, Rickettsia mooseri, Rickettsia rickettsiae, and Rickettsia conori. Other suitable bacteria may include Escherichia coli, Zymomonas mobilis, Erwinia chrysanthemi, and Klebsiella planticola.

Alternatively, parent strains can be isolated from environmental samples such as wastewater sludge from wastewater treatment facilities including municipal facilities and those at chemical or petrochemical plants. The latter are especially attractive as the isolated microorganisms can be expected to have evolved over the course of numerous generations in the presence of high product concentrations and thereby have already attained a level of desired product tolerance that may be further improved upon.

Parent strains may also be isolated from locations of natural degradation of naturally occurring feedstocks and compounds (e.g., a woodpile, a saw yard, under fallen trees, landfills). Such isolates may be advantageous since the isolated microorganisms may have evolved over time in the presence of the feedstock and thereby have already attained some level of conversion and tolerance to these materials that may be further improved upon.

Individual species or mixed populations of species can be isolated from environmental samples. Isolates, including microbial consortiums can be collected from numerous environmental niches including soil, rivers, lakes, sediments, estuaries, marshes, industrial facilities, etc. In some embodiments, the microbial consortiums are strict anaerobes. In other embodiments, the microbial consortiums are obligate anaerobes. In some embodiments, the microbial consortiums are facultative anaerobes. In still other embodiments, the microbial consortiums do not contain species of Enterococcus or Lactobacillus.

When mixed populations of specific species or genera are used, a selective growth inhibitor for undesired species or genera can be used to prevent or suppress the growth of these undesired microorganisms.

In some embodiments, the microorganisms are obligate anaerobes. Non-limiting examples of obligate anaerobes include Butyrivibrio fibrosolvens and Clostridium species.

In other embodiments, the microorganisms are microaerotolerant and are capable of surviving in the presence of small concentrations of oxygen. In some embodiments, microaerobic conditions include, but are not limited, to fermentation conditions produced by sparging a liquid media with a gas of at least about 0.01% to at least 5% or more O₂ (e.g., 0.01%, 0.05%, 0.10%, 0.50%, 0.60%, 0.70%, 0.80%, 1.00%, 1.20%, 1.50%, 1.75%, 2.0%, 3%, 4%, 5% or more O₂). In another aspect, the microaerobic conditions include, but are not limited to, culture conditions with at least about 0.05 ppm dissolved O₂ or more (e.g., 0.05, 0.075, 0.1, 0.15, 0.2, 0.3, 0.4, 0.5, 0.6, 0.8, 1.0, 2.0, 3.0, 4.0, 5.0, 8.0, 10.0, ppm or more).

In other embodiments, the microorganisms are aerotolerant.

### Culture Conditions

Optimal culture conditions for various industrially important microorganisms are known in the art. As required, the culture conditions may be anaerobic, microaerotolerant, or aerobic. Aerobic conditions are those that contain oxygen dissolved in the media such that an aerobic culture would not be able to discern a difference in oxygen transfer with the additional dissolved oxygen, and microaerotolerant conditions are those where some dissolved oxygen is present at a level below that found in air or air saturated solutions and frequently below the detection limit of standard dissolved oxygen probes, e.g., less than 1 ppm. The cultures can be agitated or left undisturbed. Typically, the pH of the media changes over time as the microorganisms grow in number, consume feedstock and excrete organic acids. The pH of the media can be modulated by the addition of buffering compounds to the initial fermentation media in the bioreactor or by the active addition of acid or base to the growing culture to keep the pH in a desired range. Growth of the culture may be monitored by measuring the optical density, typically at a wavelength of 600 nm, or by other methods known in the art.

Clostridium fermentations are generally conducted under anaerobic conditions. For example, ABE fermentations by C. acetobutylicum are typically conducted under anaerobic conditions at a temperature in the range of about 25° C. to about 40° C. Historically, suspension cultures did not use agitators, but relied on evolved or sparged gas to mix the contents of the bioreactors. Cultures, however, can be agitated to ensure more uniform mixing of the contents of the bioreactor. For immobilized cultures, a bioreactor may be run without agitation in a fixed bed (plug flow) or fluidized/expanded bed (well-mixed) mode. Thermophilic bacterial fermentations can reach temperatures in the range of about 50° C. to about 80° C. In some embodiments, the temperature range is about 55° to about 70° C. In some embodiments, the temperature range is about 60° C. to about 65° C. For example, Clostridium species such as C. thermocellum or C. thermohydrosulfuricum may be grown at about 60° C. to about 65° C. The pH of the Clostridium growth medium can be modulated by the addition of buffering compounds to the initial fermentation media in the bioreactor or by the active addition of acid or base to the growing culture to keep the pH in a desired range. For example, a pH in the range of about 3.5 to about 7.5, or about 5 to about 7, may be maintained in the medium for growth of Clostridium.

### Immobilization of Microorganisms on Solid or Semi-Solid Support

Optionally, microorganisms are grown immobilized on a solid or semi-solid support for production of one or more bioproduct(s) of interest.

Immobilization of the microorganism, from spores or vegetative cells, can be by any known method. In one embodiment, entrapment or inclusion in the support is achieved by polymerizing or solidifying a spore or vegetative cell containing solution. Useful polymerizable or solidifiable solutions include, but are not limited to, alginate, κ-carrageenan, chitosan, polyacrylamide, polyacrylamide-hydrazide, agarose, polypropylene, polyethylene glycol, dimethyl acrylate, polystyrene divinyl benzene, polyvinyl benzene, polyvinyl alcohol, epoxy carrier, cellulose, cellulose acetate, photocrosslinkable resin, prepolymers, urethane, and gelatin.

In another embodiment, the microorganisms are incubated in growth medium with a support. Useful supports include, but are not limited to, bone char, cork, clay, resin, sand, porous alumina beads, porous brick, porous silica, celite (diatomaceous earth), polypropylene, polyester fiber, ceramic, (e.g., porous ceramic, such as porous silica/alumina composite), lava rock, vermiculite, ion exchange resin, coke, natural porous stone, macroporous sintered glass, steel, zeolite, engineered thermal plastic, concrete, glass beads, Teflon, polyetheretherketone, polyethylene, wood chips, sawdust, cellulose fiber (pulp), or other natural, engineered, or manufactured products. The microorganisms may adhere to the support and form an aggregate, e.g., a biofilm.

In another embodiment, the microorganism is covalently coupled to a support using chemical agents like glutaraldehyde, o-dianisidine (U.S. Pat. No. 3,983,000), polymeric isocyanates (U.S. Pat. No. 4,071,409), silanes (U.S. Pat. Nos. 3,519,538 and 3,652,761), hydroxyethyl acrylate, transition metal-activated supports, cyanuric chloride, sodium periodate, toluene, or the like. See also U.S. Pat. Nos. 3,930,951 and 3,933,589.

In one embodiment, immobilized spores, such as those of Clostridium, e.g., C. acetobutylicum, are activated by thermal shock and then incubated under appropriate conditions in a growth medium whereby vegetative growth ensues. These cells remain enclosed in or on the solid support. After the microorganisms reach a suitable density and physiological state, culture conditions can be changed for bioproduct production. If the immobilized cells lose or exhibit reduced bioproduct production ability, they can be reactivated by first allowing the cells to sporulate before repeating the thermal shock and culture sequence.

Vegetative cells can be immobilized in different phases of their growth. For microorganisms that display a biphasic culture, such as C. acetobutylicum with its acidogenic and solventogenic phases, cells can be immobilized after they enter the desired culture phase in order to maximize production of the desired products, where in the case of C. acetobutylicum it is the organic acids acetic acid and butyric acid in the acidogenic phase and the solvents acetone, butanol and ethanol in the solventogenic phase. Alternatively, biphasic cells can be immobilized in the acidogenic phase and then adapted for solvent production.

In some embodiments, microorganisms to be immobilized in a bioreactor are introduced by way of a cell suspension. Generally, these microorganisms are dispersed in the media as single cells or small aggregates of cells. In other embodiments, the microorganisms are introduced into the bioreactor through the use of suspended particles that are colonized by the microorganisms. These suspended particles can be absorbed onto the solid support and frequently are of sufficiently small size that they can enter and become immobilized in the pore structures of the solid support. Typically, regardless of the suspended particle size, microorganisms can be transferred by contact with the solid support. A biofilm on the introduced particles can transfer to and colonize these new surfaces. In some embodiments, the desired characteristics of the microorganisms can only be maintained by culturing on a solid support, thereby necessitating the use of small colonized particle suspensions for seeding a solid support in a bioreactor.

### Support for Immobilized Microbial Growth

In some embodiments, a bioproduct producing microorganism is grown in an immobilized form on a solid or semi-solid support material in a bioreactor as described herein. In some embodiments, the support contains a porous material. Non-limiting examples of suitable support materials include bone char, synthetic polymers, natural polymers, inorganic materials, and organic materials.

Natural polymers include organic materials such as cellulose, lignocellulose, hemicellulose, and starch. Organic materials include feedstock such as plant residue and paper. Composites of two or more materials may also be used such as mixtures of synthetic polymer with natural plant polymer.

Examples of semi-solid media include alginate, α-carrageenan and chitosan, polyacrylamide, polyacrylamide-hydrazide, agarose, polypropylene, polyethylene glycol, dimethyl acrylate, polystyrene divinyl benzene, polyvinyl benzene, polyvinyl alcohol, epoxy carrier, cellulose, cellulose acetate, photocrosslinkable resin, prepolymers, urethane, and gelatin. Examples of solid support include cork, clay, resin, sand, porous alumina beads, porous brick, porous silica, celite, wood chips or activated charcoal.

Suitable inorganic solid support materials include inorganic materials with available surface hydroxy or oxide groups. Such materials can be classified in terms of chemical composition as siliceous or nonsiliceous metal oxides. Siliceous supports include, inter alia, glass, colloidal silica, wollastonite, cordierite, dried silica gel, bentonite, and the like. Representative nonsiliceous metal oxides include alumina, hydroxy apatite, and nickel oxide.

In some embodiments, the support material is selected from bone char, polypropylene, steel, diataomaceous earth, zeolite, ceramic, (e.g., porous ceramic, such as porous silica/alumina composite), engineered thermal plastic, clay brick, concrete, lava rock, wood chips, polyester fiber, glass beads, Teflon, polyetheretherketone, polyethylene, vermiculite, ion exchange resin, cork, resin, sand, porous alumina beads, coke, natural porous stone, macroporous sintered glass, or a combination thereof. In one embodiment, the support material is bone char. Useful support material has a high surface area to volume ratio such that a large amount of active, productive cells can accumulate in the bioreactor. Useful supports may contain one or more macrostructured components containing one or more useful support material(s) that promotes good fluidmechanical properties, for example, a wire mesh/gauze packing material used for traditional distillation tower packing.

In some embodiments, the support material is chosen to support growth of the fermenting bioproduct producing microorganism as a biofilm. The biofilm may grow on exterior surfaces of support particles, in the fluid space between support particles, and/or on surfaces in the interior of pores of the support material.

### Continuous Process

In some embodiments, a continuous process for bioproduct production is provided. In a continuous production process herein, a carbohydrate-containing composition, for example, liquid sugar-containing extract (e.g., cane juice and/or molasses) and/or a hydrolysate or conditioned hydrolysate of residual biomass (e.g., bagasse and/or cane straw) from the sugar production facility is continuously fed to one or more bioreactors for microbial production of the bioproduct, the bioproduct is continuously produced by immobilized microorganism(s) in the one or more bioreactors, and bioproduct-containing effluent, i.e., fermentation broth, is continuously withdrawn from the one or more reactors, for the duration of fermentation. In some embodiments, feedstock (e.g., bagasse and/or cane straw) is continuously hydrolyzed to release soluble sugar molecules, and continuously conditioned prior to introduction of the conditioned hydrolyzed feedstock into the bioreactor(s). The conditioning process may operate continuously downstream from a feedstock hydrolysis process, and upstream from the bioreactor(s), and conditioned hydrolyzed feedstock may be continuously fed to the bioreactor for the duration of fermentation. In some embodiments, the microorganism is tolerant to inhibitors and/or the hydrolyzed feedstock does not contain substances that are inhibitory to the microorganism that is used for bioproduct production and conditioning is not required.

In some embodiments, the continuous process may also include downstream continuous concentration and/or purification processes for recovery of the bioproduct, wherein continuously withdrawn effluent is continuously processed in one or more concentration and/or purification processes to produce a bioproduct.

In some embodiments, the process may also include deconstruction of the feedstock and/or removal of extractives from the feedstock, as described herein. Deconstruction and/or removal of extractives may be continuous or may occur prior to or periodically throughout the continuous process.

In some embodiments, the process operates continuously for at least about 50, 100, 200, 300, 400, 600, 800, 1000, 1350, 1600, 2000, 2500, 3000, 4000, 5000, 6000, 7000, 8000, or 8400 hours.

A “continuous” process as described herein may include periodic or intermittent partial or complete shutdowns of one or more parts of the bioproduct production system for processes such as maintenance, repair, regeneration of resin, etc.

Continuous fermentation, with constant feed of feedstock and withdrawal of product-containing microbial broth, can minimize the unproductive portions of a fermentation cycle, such as lag, growth, and turnaround time, thereby reducing the capital cost, and can reduce the number of inoculation events, thus minimizing operational costs and risk associated with human and process error.

The continuous methods and systems described herein can utilize one or more, e.g., one, two, or three or more, bioreactors. When multiple (two or more) bioreactors are used, they may be arranged in parallel, series, or a combination thereof. The bioreactors can grow the same or different strains of microorganism(s).

In other embodiments, a batch process is used, with shorter fermentation times, e.g., about 10 hours to about 100 hours, about 12 hours to about 24 hours, about 20 hours to about 30 hours, about 24 hours to about 48 hours, about 48 hours to about 72 hours, about 72 hours to about 96 hours, about 70 hours to about 100 hours, or about 10 hours, about 12 hours, about 20 hours, about 24 hours, about 30 hours, about 40 hours, about 48 hours, about 50 hours, about 60 hours, about 70 hours, about 72 hours, about 80 hours, about 90 hours, about 96 hours, or about 100 hours.

### Integration and Recycling of Process Streams and Biomaterials

In some embodiments, process streams and/or biomaterial from the sugar processing facility, bioproduct production facility, and/or optional ethanol production facility may be used to provide fluids and/or nutrients for bioproduct production, energy for operation of the integrated biorefinery system as disclosed herein, and/or energy for other uses, for example, to the electricity grid.

In one embodiment, steam that is used for processing of sugar cane in the sugar processing facility is recovered and used for heat for the fermentation process in the bioproduct production facility.

In one embodiment, a condensate is prepared from steam that is used for processing of sugar cane in the sugar processing facility. The condensate may be provided as liquid in the microorganism growth medium for production of the bioproduct(s) in the bioproduct production facility.

In one embodiment, vinasse is prepared by removal of bioproduct(s) from the fermentation broth in the bioproduct production facility and/or by removal of ethanol from the fermentation broth in the optional ethanol production facility. Bioproduct (e.g., butanol) vinasse and/or ethanol vinasse may be used as liquid in the microorganism growth medium for production of bioproduct(s) in the bioproduct production facility. Vinasse may also or alternately be used to remove residual sugar from biomass (e.g., bagasse and/or cane straw) after pretreatment (hydrolysis). Such residual sugar removal may, for example, include washing of the biomass with vinasse, e.g., butanol or ethanol vinasse. In one embodiment, a countercurrent cascade washing procedure is used. The liquid containing vinasse and removed residual sugar may be provided to the fermentation medium in the bioproduct production facility for production of bioproduct(s).

In embodiments which include an ethanol production facility, spent yeast from ethanol production may be recovered. For example, yeast cells may be recovered from distillation stillage. For example, cell mass is recovered from the bottom of an ethanol distillation column. In some embodiments, the stillage is evaporated to form a thick concentrate called “yeast cream.” The yeast cells may be provided to the fermentation medium in the bioproduct production facility and/or may be included in hydrolysis mixture for the preparation of biomass (e.g., bagasse and/or cane straw) hydrolysate. In one embodiment, the yeast cells provide nutrients for growth of the bioproduct-producing microorganisms and/or production of bioproduct(s) in the bioproduct production facility.

In some embodiments, residual solid material remaining after hydrolysis of biomass (e.g., bagasse and/or cane straw) and separation of liquid from solid material as described herein is recovered and used as an energy source. In some embodiments, the residual solid material contains primarily cellulose and lignin. In other embodiments, the residual solid material contains primarily lignin. The solid material may be used as a fuel source. For example, it may be used as a fuel source for one or more boiler(s) that provide heat for the fermentation process in the bioproduct production facility. It may also or alternately be used to produce electricity for use within the integrated biorefinery or to provide electricity to the electricity grid. In various embodiments, the material may be burned to produce heat which is used directly (e.g., flue gas for drying) or indirectly (e.g., steam for direct or indirect use, such for electricity generation by a turbine). In some embodiments, nitrate (e.g., NOₓ) is removed from residual solid material prior to use as a fuel source. In some embodiments, nitrate is removed by washing with water, caustic, or vinasse (e.g., ethanol and/or bioproduct (e.g., butanol) vinasse). For example, residual solid material is provided in which nitrate levels have been reduced by about 90%, about 95%, or up to 100% or essentially 100%, in comparison to the material prior to nitrate removal. Wash water that contains nitrate may optionally be denitrified, for example, by a denitrifying microorganism (see, e.g., U.S. Pat. No. 6,019,900). The solid residual material may also be used to supply lignin and/or cellulose for production of downstream lignin and/or cellulose based or derived products.

### Compositions

Compositions are provided herein that include materials or process streams from an integrated biorefinery system as described herein.

In some embodiments, fermentation media are provided that include material from one or more process streams from the integrated biorefinery. In one embodiment, a fermentation medium is provided that includes a liquid sugar-containing extract (e.g., cane juice; molasses) from a sugar processing facility and sugar molecules extracted from residual biomass (e.g., bagasse; cane straw) after sugar processing in the sugar processing facility. In another embodiment, the fermentation medium contains cane juice and/or molasses from sugar cane and also contains a hydrolysate of bagasse and/or cane straw.

In some embodiments, the fermentation medium contains vinasse. In one embodiment, the fermentation medium contains butanol vinasse. In another embodiment, the fermentation medium contains ethanol vinasse. In one embodiment, the fermentation medium contains a liquid sugar-containing extract (e.g., cane juice; molasses) from a sugar processing facility and sugar molecules extracted from residual biomass (e.g., bagasse; cane straw) after sugar processing in the sugar processing facility, and further contains vinasse (e.g., butanol and/or ethanol vinasse).

In some embodiments, the fermentation medium contains steam condensate from a sugar processing facility. In some embodiments, the fermentation medium contains spent yeast cells from an ethanol production facility.

In various embodiments, fermentation media are provided that include one or more of: a liquid sugar-containing extract (e.g., cane juice; molasses) from a sugar processing facility; sugar molecules extracted from residual biomass (e.g., bagasse; cane straw) after sugar processing in the sugar processing facility, such as a hydrolysate of bagasse and/or a hydrolysate of cane straw; vinasse (e.g., butanol and/or ethanol vinasse); steam condensate from sugar processing in a sugar processing facility; and spent yeast cells from an ethanol production facility.

In some embodiments, the fermentation medium contains about 5% to about 85% vinasse (v/v) (e.g., butanol vinasse and/or ethanol vinasse). In some embodiments, the fermentation medium contains about 5%, about 10%, about 15%, about 20%, about 25%, about 30%, about 35%, about 40%, about 45%, about 50%, about 55%, about 60%, about 65%, about 70%, about 75%, about 80%, or about 85% vinasse (e.g., butanol vinasse and/or ethanol vinasse).

In some embodiments, the fermentation medium contains about 40% to about 90% (v/v) biomass hydrolysate (e.g., hydrolysate of bagasse and/or cane straw). In various embodiments, the fermentation medium may contain any of about 40%, 45%, 50%, 55%, 60%, 65%, 70%, 75%, 80%, 85%, or 90% hydrolysate of bagasse, hydrolysate of cane straw, or a combination of bagasse and cane straw hydrolysate.

In some embodiments, the fermentation medium contains about 0.1% to about 20% (v/v) liquid sugar-containing stream from a sugar production facility (e.g., cane juice and/or molasses). In various embodiments, the fermentation medium may contain any of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% cane juice, molasses, or a combination of cane juice and molasses.

In some embodiments, the fermentation medium contains about 40% to about 90% (v/v) biomass hydrolysate (e.g., hydrolysate of bagasse and/or cane straw), about 0.1% to about 20% (v/v) cane juice and/or molasses, and about 9.9% to about 60% (v/v) vinasse (e.g., butanol vinasse and/or ethanol vinasse). In various embodiments, the fermentation medium may contain any of about 40%, 45%, 50%, 55%, 60%, 65%, 70%, 75%, 80%, 85%, or 90% hydrolysate of bagasse, hydrolysate of cane straw, or a combination of bagasse and cane straw hydrolysate; any of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% cane juice, molasses, or a combination of cane juice and molasses; and any of about 9.9%, 10%, 15%, 20%, 25%, 30%, 35%, 40%, 45%, 50%, 55%, or 60% vinasse (e.g., butanol vinasse, ethanol vinasse, or a combination of butanol vinasse and ethanol vinasse).

The fermentation medium may contain biomass hydrolysate that includes bagasse hydrolysate and cane straw hydrolysate in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0. The fermentation medium may contain cane juice and molasses in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0. The fermentation medium may contain butanol vinasse and ethanol vinasse in any percent ratio, such as, for example, 0:100, 5:95, 10:90, 15:85, 20:80, 25:75, 30:80, 35:65, 40:60, 45:55, 50:50, 55:45, 60:40, 65:35, 70:30, 75:25, 80:20, 85:15, 90:10, 95:5, and 100:0.

In some embodiments, biomass materials are provided from which nitrates have been reduced or removed. In one embodiment, bagasse and/or cane straw is provided from which nitrates have been reduced or removed. In another embodiment, residual solid material remaining after biomass pretreatment (e.g., residual solids after hydrolysis of bagasse and/or cane straw) is provided from which nitrates have been reduced or removed. For example, residual solid material is provided in which nitrate levels have been reduced by about 90%, about 95%, or up to 100% or essentially 100%, in comparison to the material prior to nitrate removal.

### Exemplary Embodiments of Integrated Systems and Methods for Bioproduct Production

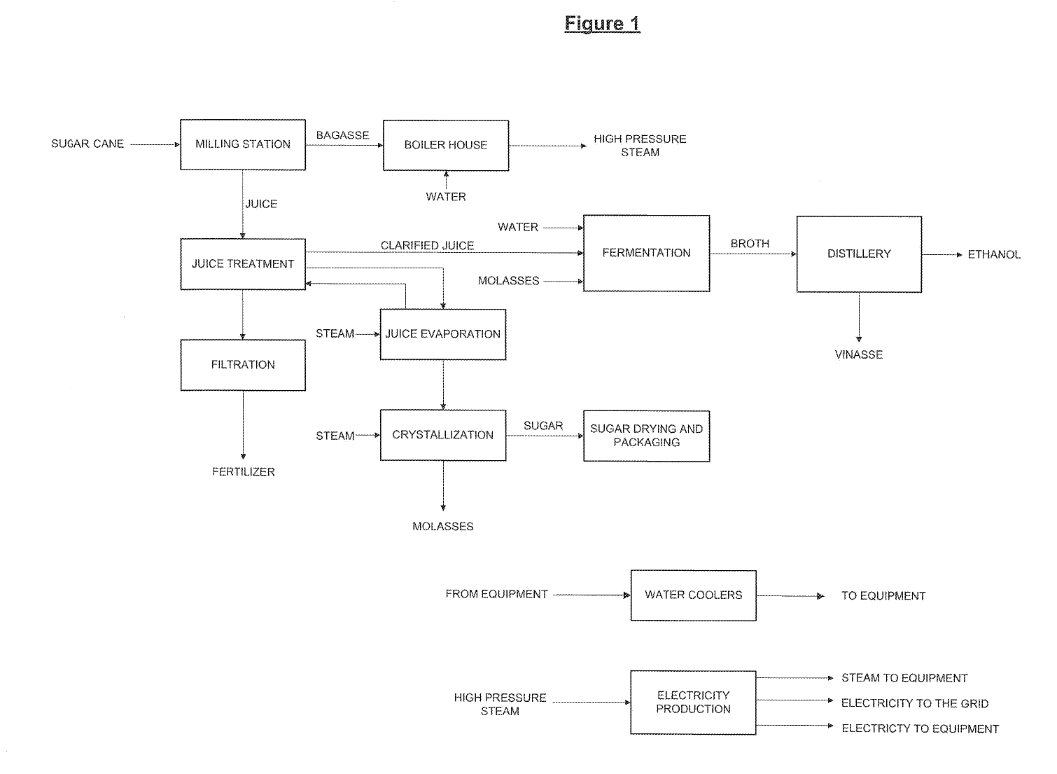

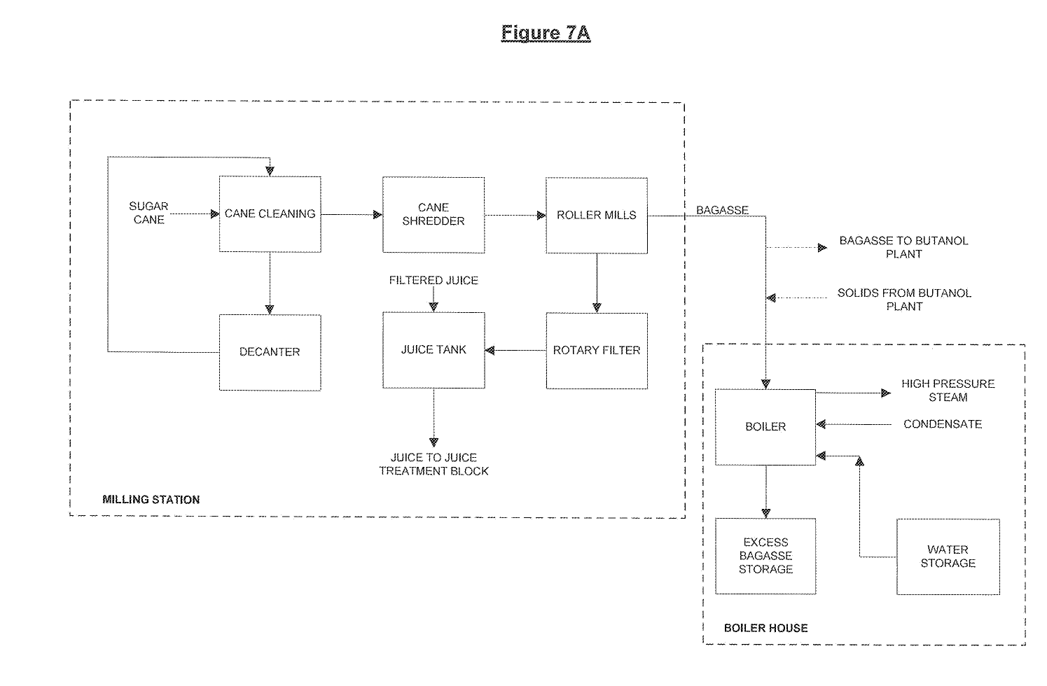

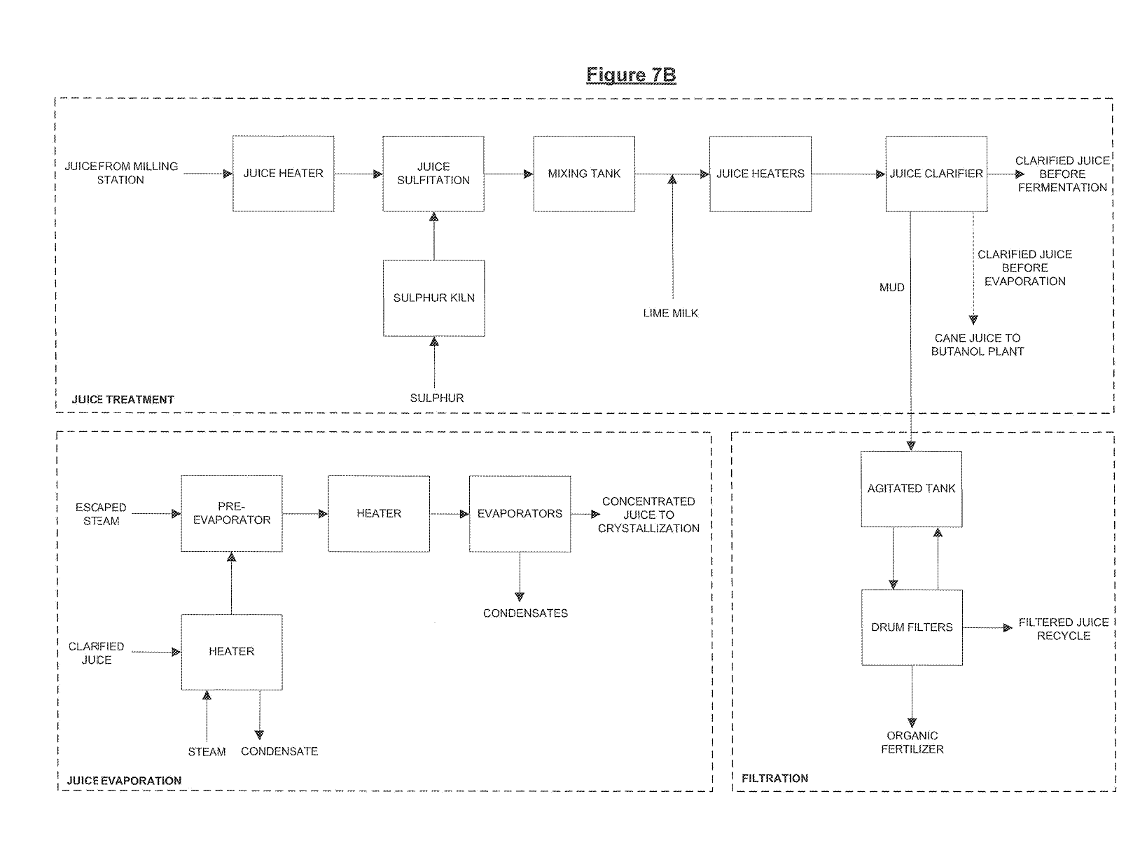

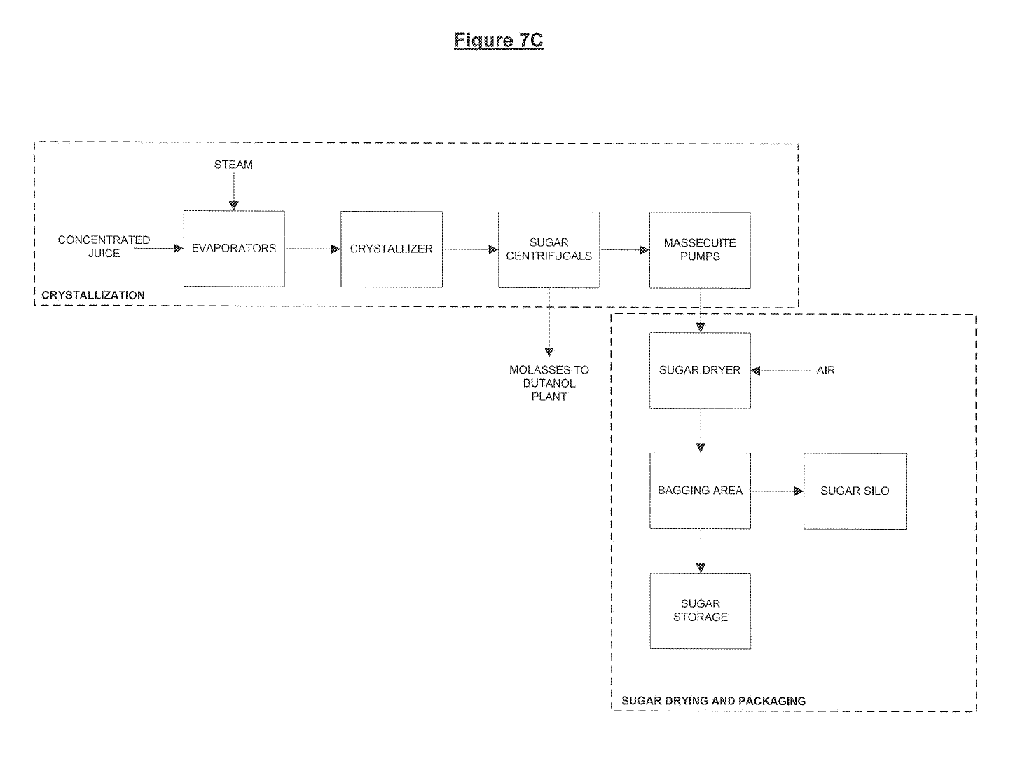

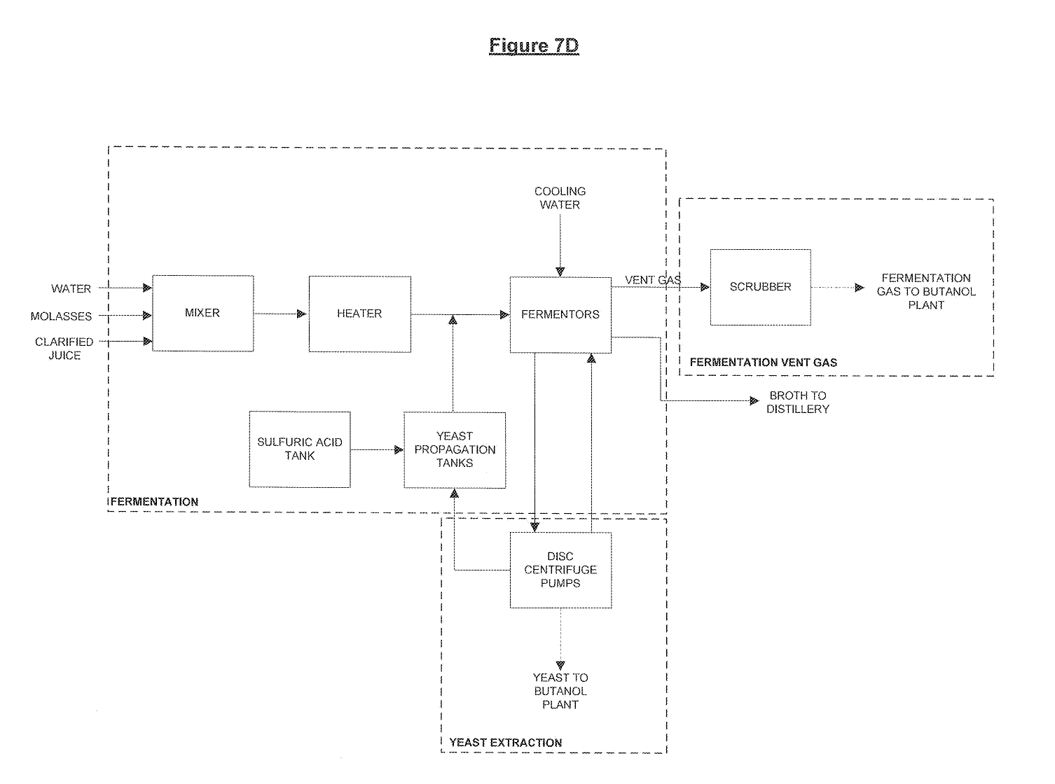

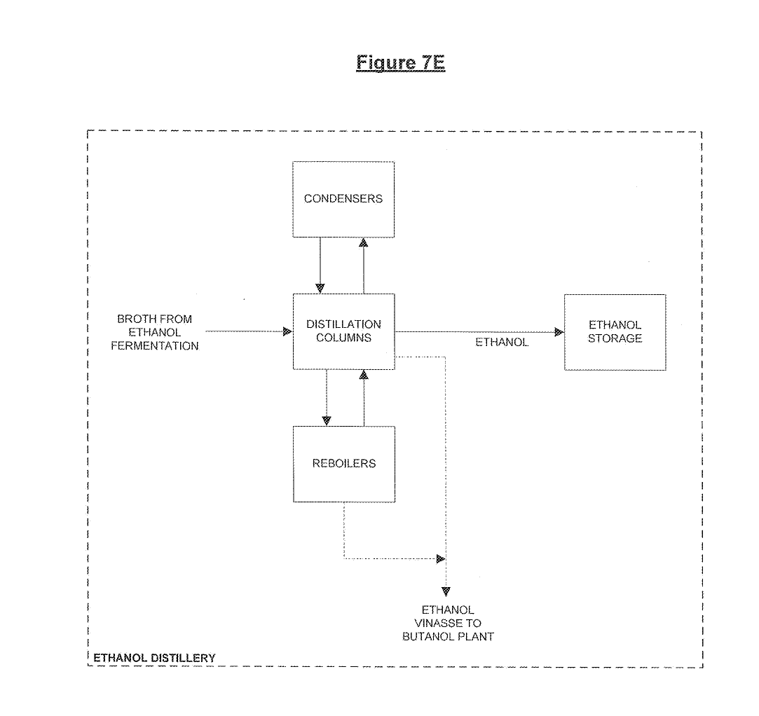

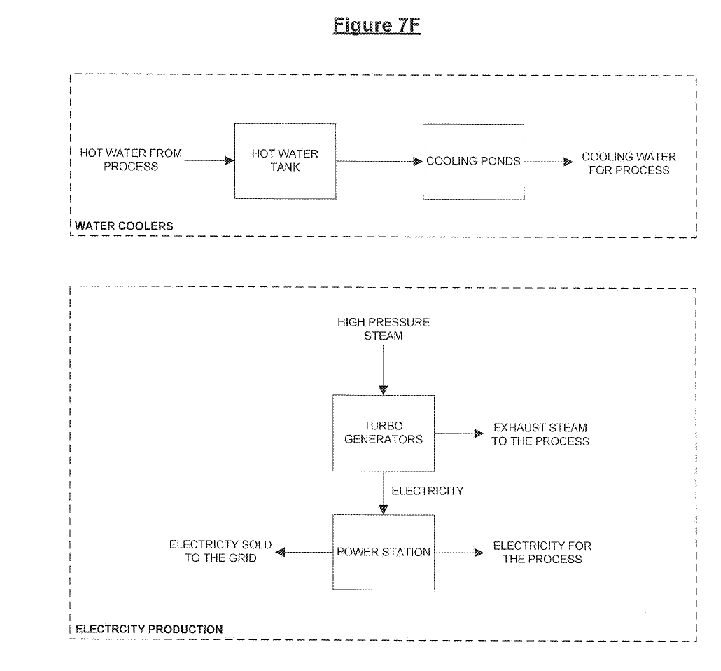

Referring to FIG. 1 and FIGS. 7A through 7F, a schematic diagram of an exemplary embodiment of a sugar production facility (i.e. sugar mill) with an integrated ethanol production facility (i.e. distillery or plant) is shown.

The sugar cane is delivered to the sugar mill where it goes through a cleaning step followed by milling, cane juice treatment, juice evaporation, sugar crystallization, sugar drying, packaging and shipping. The milling of the sugar cane is accomplished by a multi-stage process (shown as employing a series of rollers). According to an exemplary embodiment, at an initial stage juice is extracted from the sugar cane; the juice is used for sugar production. At successive stages juice and then molasses is extracted. Juice and molasses from the later stages can be used as a feedstock for the production of ethanol at the ethanol production facility. Residual molasses is also available for use as a co-product, including for animal feed applications or for sale to beverage alcohol producers. At the final stages, a biomass material referred to as bagasse is the residue. Bagasse may be used as a primary feedstock for the production of one or more bioproduct(s) such as butanol.

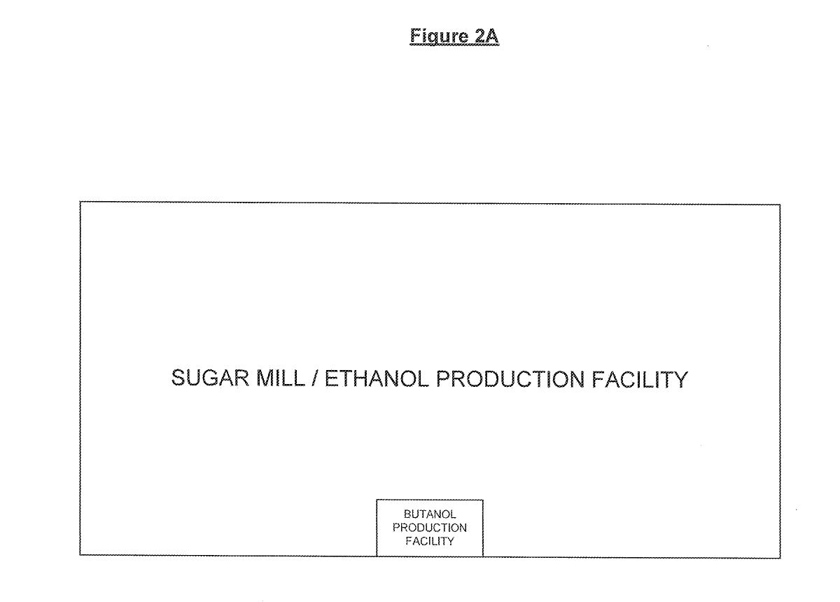

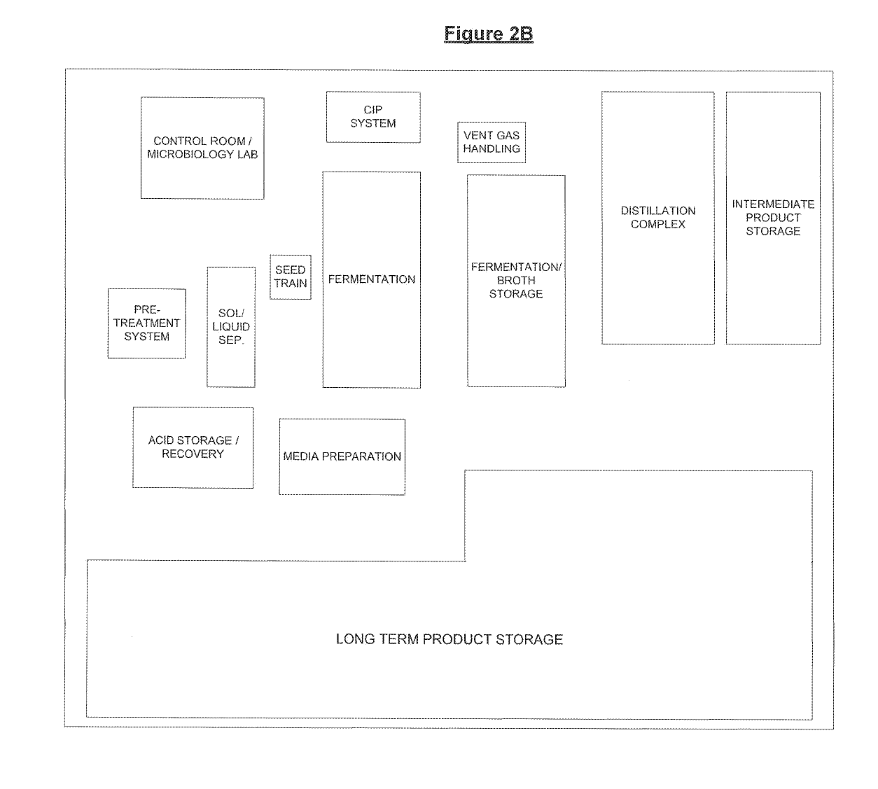

Referring to FIG. 2A, a sugar mill with an integrated ethanol production facility is shown with a co-located/adjacent butanol production facility.

Referring to FIG. 2B, a schematic diagram of an exemplary embodiment of a butanol production facility (i.e. butanol plant) is shown. According to a preferred embodiment, the butanol plant is configured to produce butanol (and co-products) from biomass. The butanol plant comprises a number of plant operations and functions, as shown in FIG. 2B.

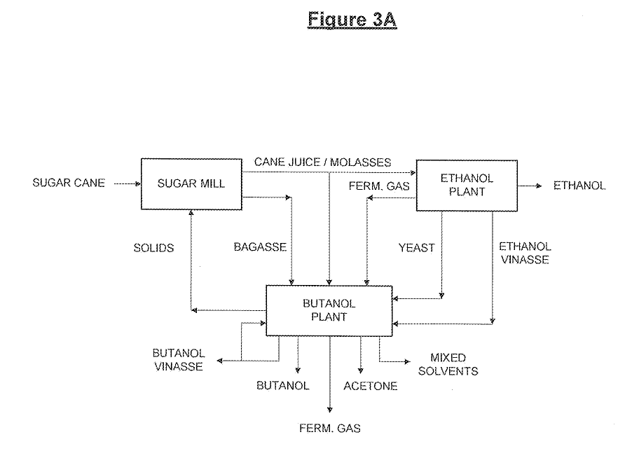

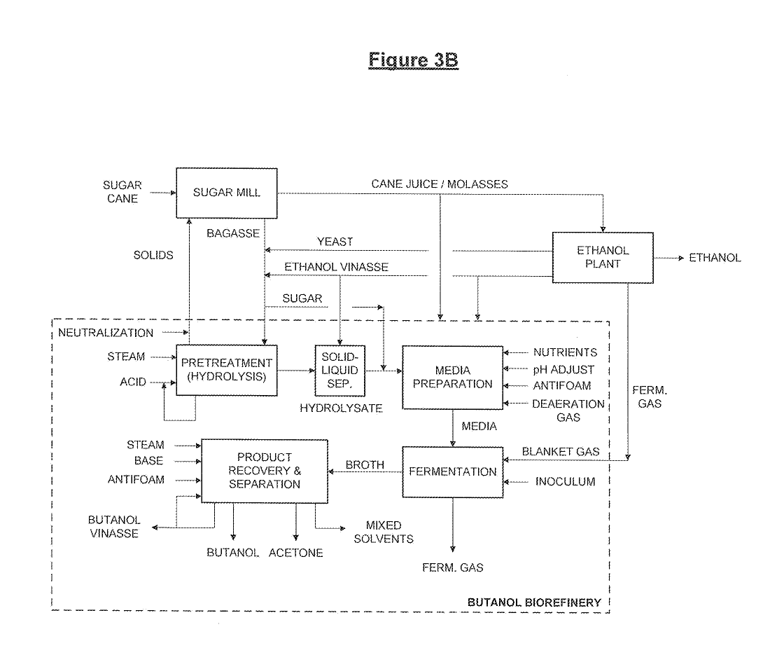

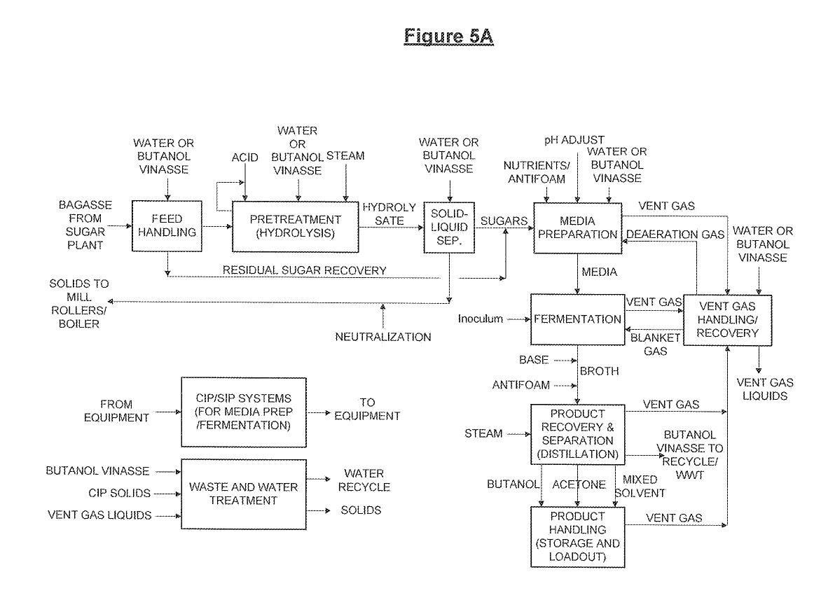

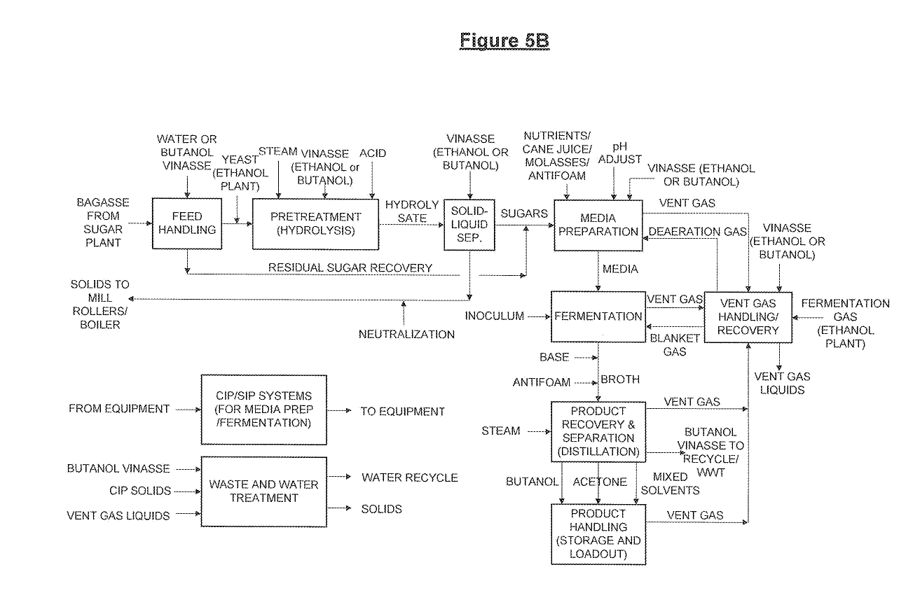

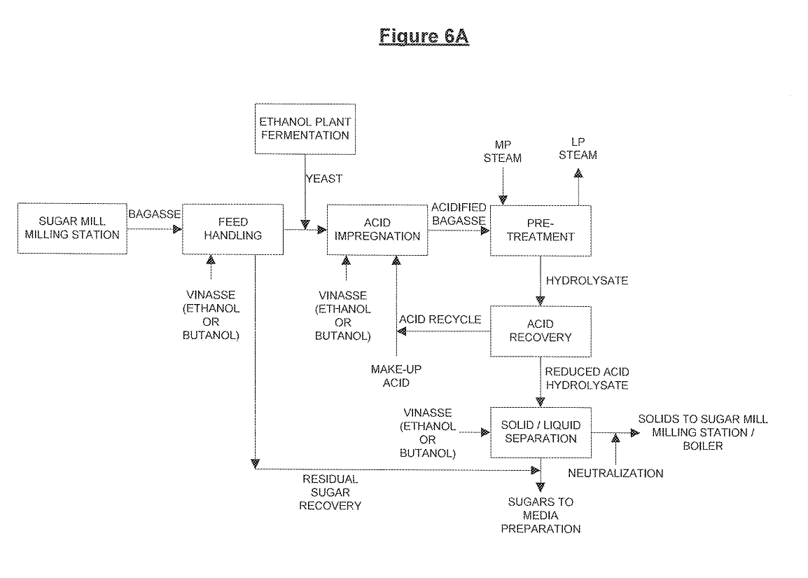

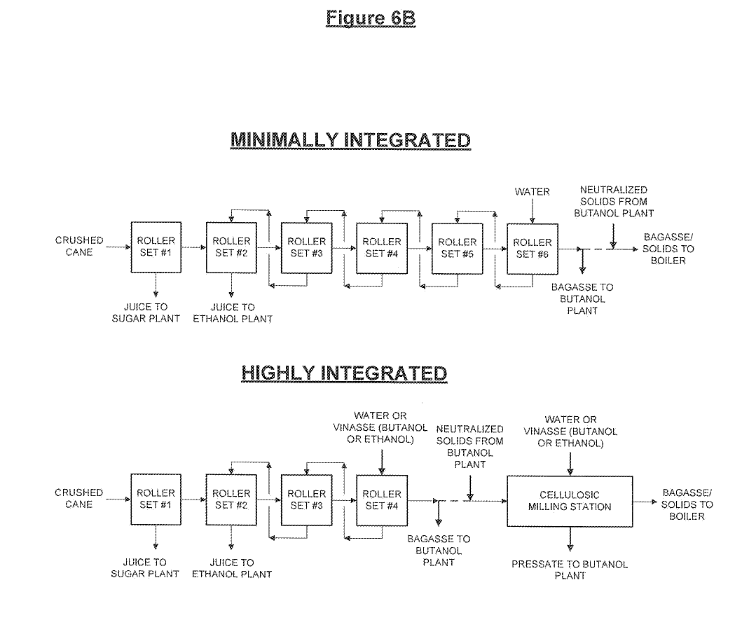

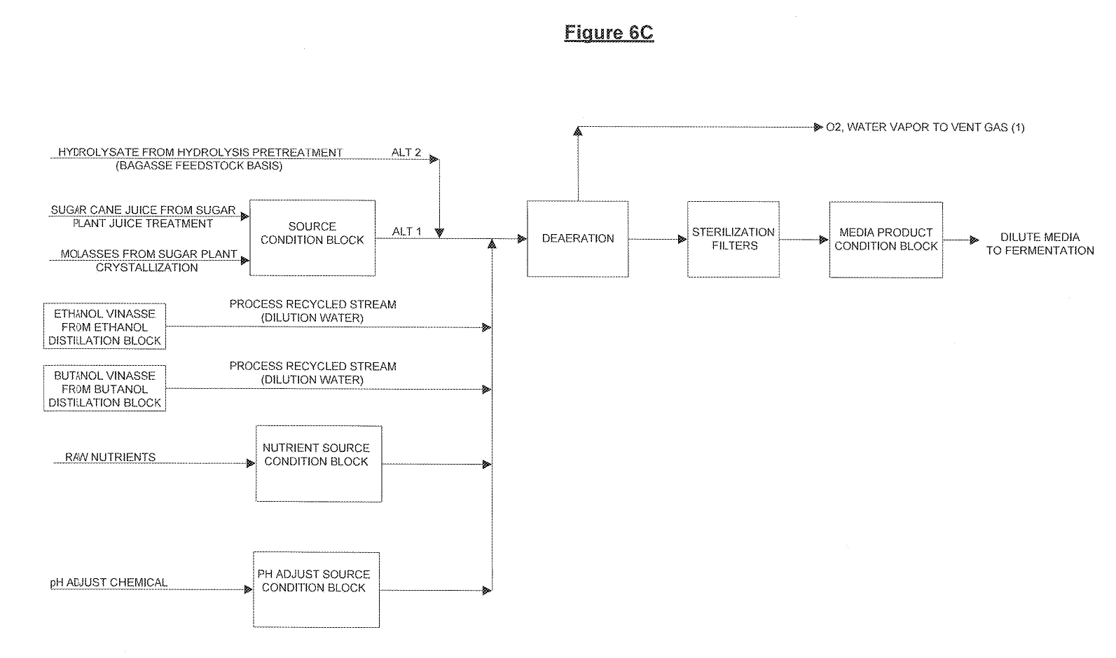

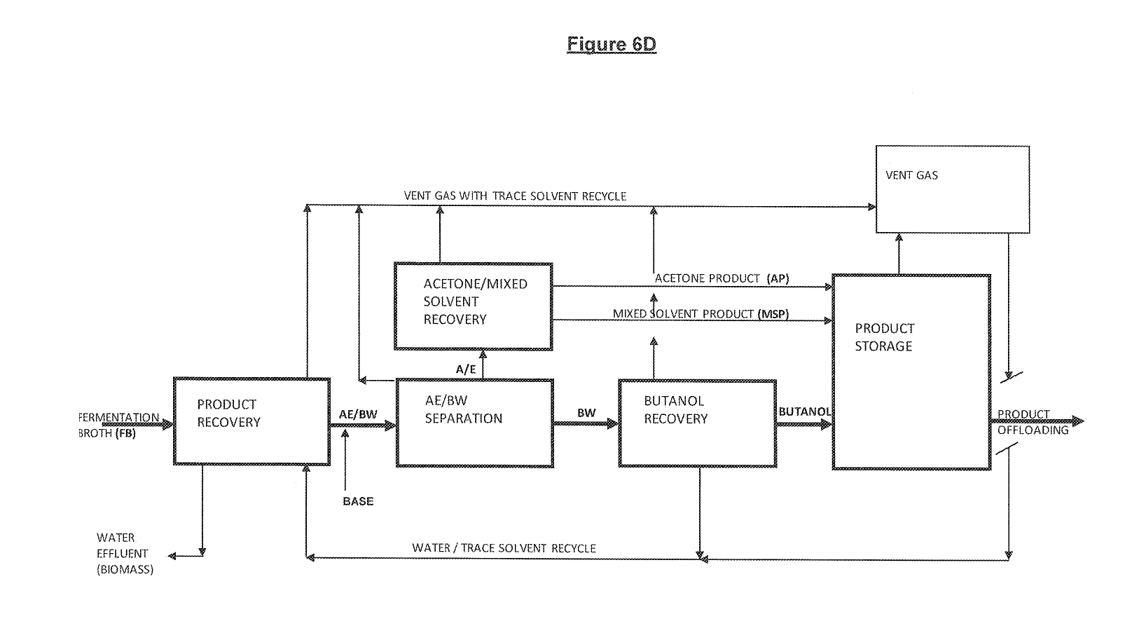

Referring to FIG. 3A, a sugar mill with an integrated ethanol production facility (ethanol plant) and an integrated butanol production facility (butanol plant) is shown according to an exemplary embodiment. As shown in FIGS. 3A and 3B (system diagrams) and 4A and 4B (showing tie-in points), integration of the three plants is accomplished by interconnection of process streams and plant inputs/outputs. Bagasse is supplied from the sugar mill to the butanol plant and pre-treated into a hydrolysate that contains sugars (e.g. C5 sugars/hemicellulose) that can supplied to the fermentation system to produce butanol. As shown in FIGS. 5B and 6A, the bagasse may also contain residual sugars (e.g. sucrose) that can be removed from the bagasse by conditioning (e.g. washing); the residual sugars may be supplied to the fermentation process and used to produce butanol. Cane juice and molasses may also be supplied from the sugar mill to the butanol plant and/or the ethanol plant (e.g. to the fermentation system and used to produce butanol or ethanol, respectively, at each plant). Ethanol vinasse, yeast and fermentation gas is supplied from the ethanol plant to the butanol plant. Ethanol vinasse can be used as a substitute or supplement for process water (after conditioning); the ability to use vinasse as process water reduces the overall water consumption of the integrated biorefinery. Yeast from the ethanol plant may be used as a nutrient source for the organism used to produce butanol. (Vinasse may also contain nutrients for the organism.) Fermentation gas may be used as a blanket gas in the fermentation system of the butanol plant. Biomass residue (e.g. C6/cellulosic solids) are supplied to the sugar mill (e.g. as an energy source/fuel). Butanol vinasse may be recycled/re-used in the butanol plant. The sugar mill will produce refined sugar and other sugar products and co-products (including molasses). The ethanol plant will produce ethanol and co-products. The butanol plant will produce butanol, acetone and other mixed solvents.

FIGS. 5A (standard butanol plant) and 5B (integrated butanol plant) show the additional process streams that are available and interconnected in the integrated butanol plant of FIGS. 3A and 3B.

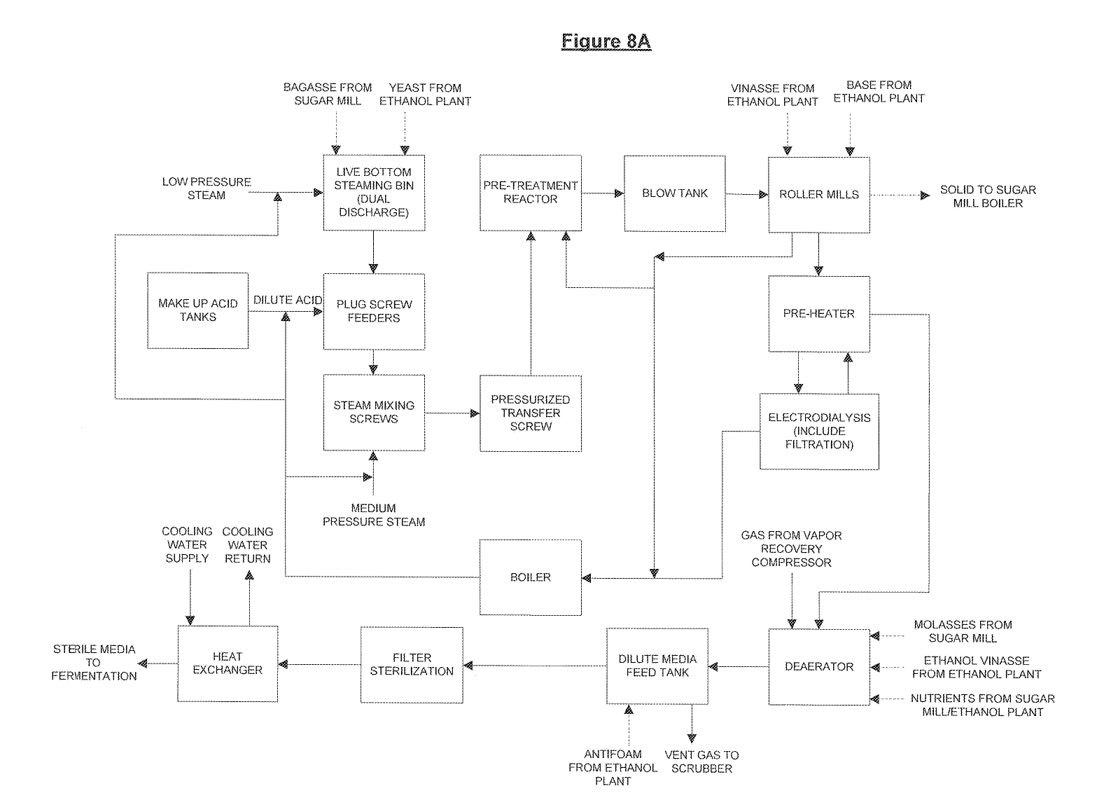

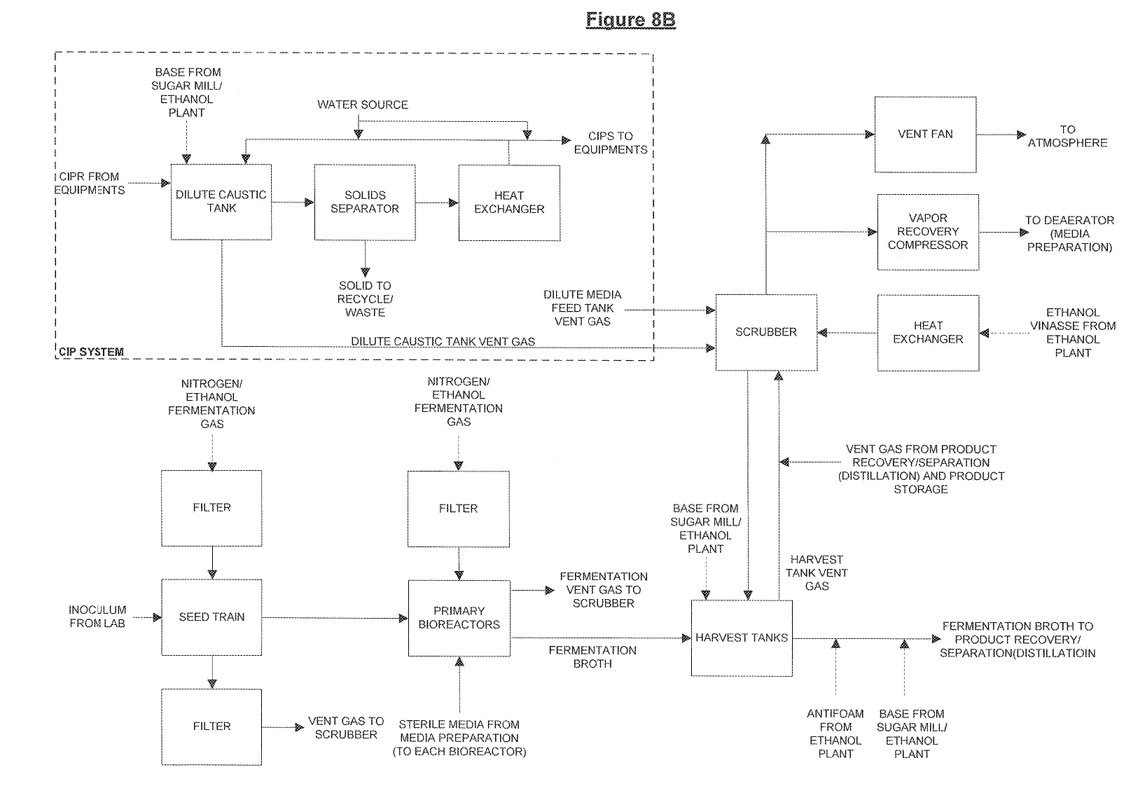

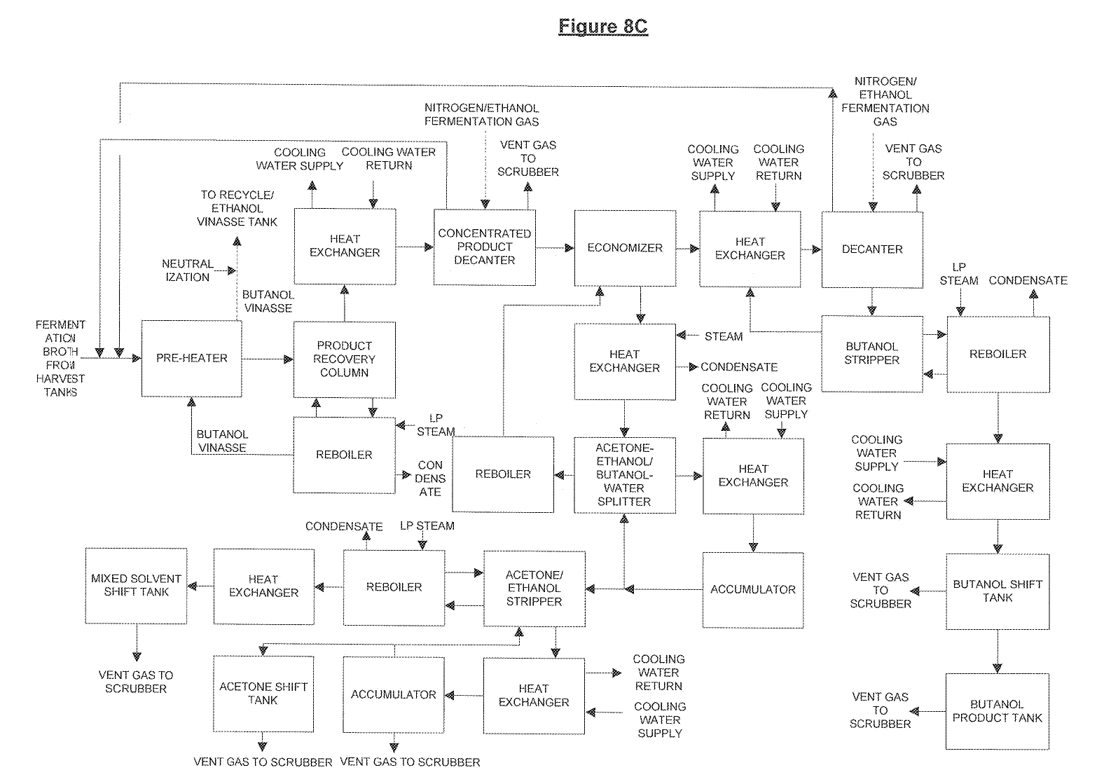

FIGS. 6A through 6D (block diagram) and FIGS. 8A through 8C (schematic diagrams) show the butanol plant according to an exemplary embodiment of the integrated biorefinery.

FIG. 6A shows the pretreatment system for biomass (e.g. bagasse) in the integrated biorefinery. FIG. 6A also shows the use of ethanol vinasse as a water source for pretreatment and solids washing to increase hydrolysate sugar recovery. Yeast is or can be added in pretreatment to provide a way to lyse the yeast cell for access to the cellular nutrients and to use the cell wall to mitigate inhibitors that may form during pretreatment. Cellulosic (C6) solids are neutralized prior to returning the material to the sugar plant for steam and electricity production. Hemicellulosic material (C5) is conditioned and supplied to the fermentation system for butanol production.

FIG. 6B shows the integration of the sugar cane milling system of the sugar mill with the ethanol plant and butanol plant according to two exemplary embodiments. As shown in the minimally integrated embodiment, the interconnections to the butanol plant may be made at existing points where the materials to be supplied to the butanol plant become available, for example with bagasse as it exists at the completion of processing of the sugar cane (in the milling system); as shown in the highly integrated embodiment, the interconnections to the butanol plant may be configured to condition the materials to be supplied to the butanol plant, for example to mix the bagasse with vinasse (as process water) for supply to the butanol plant. The bagasse may be conditioned to recover sugar (e.g. sucrose by washing) of the bagasse before it is pretreated.

FIG. 6C shows the integration of ethanol vinasse into the media preparation and fermentation system of the butanol plant. The vinasse provides important nutrients as well as water for the fermentation process; as also shown, the sugar mill also may supply other nutrient/feedstock sources, such as sugar cane juice and molasses.

FIG. 6D shows the product recovery system of the butanol plant. The fermentation system produces butanol, acetone, mixed solvents, and butanol vinasse that can recovered and/or recycled and reused in the pretreatment and fermentation processes of the integrated biorefinery. According to an alternative and/or additional embodiment, vinasse (from ethanol production and/or butanol production) can also be field-applied outside of the plant.

As shown in FIGS. 8A through 8C, other process streams may be interconnected in the integrated biorefinery, for example, anti-foam agent from the ethanol plant, solids (e.g. C6/cellulosic solids) for the solid fuel boiler of the sugar plant may be provided from the butanol plant, sodium hydroxide from the sugar mill or the ethanol plant may be supplied to the butanol plant for use in plant maintenance/cleaning (e.g. CIP/clean-in-place system), fermentation gases from the ethanol plant may be used as a blanket gas for process management or in product recovery/storage. Other process streams available within the integrated biorefinery may be used or shared for a wide variety of purposes relating to production efficiencies, material savings, waste reduction, recycling/reuse, to achieve energy efficiencies (e.g. as heat exchanger fluids), for maintenance/cleaning (e.g. as rinse water), etc.

According to any preferred embodiment, the integrated biorefinery will interconnect the plants for efficient operation and production.

According to particularly preferred embodiments of the integrated biorefinery, there will be minimal disruption of the sugar mill and current production of sugar, ethanol, and molasses. The butanol plant will be configured to selectively utilize sugar and sugar-based products or process streams from the sugar mill. For example, molasses is available as a nutrient base for the fermentation system but is not required or may be required in minimal amounts. Fresh water usage can be reduced by use and reuse of vinasse from the ethanol plant and/or butanol plant; ethanol and/or butanol vinasse that may otherwise be spread in fields (field-applied) may be used as process water for the butanol plant, reducing potentially detrimental effect on the surrounding soil. The sugar mill may use sugar cane bagasse (biomaterial left over after sugar extraction), as fuel for a solid fuel boiler for combined heat and power for the sugar mill. In the integrated biorefinery, the bagasse will provide hemicellulose sugar (C5) sugar for ethanol production, leaving the cellulose and lignin (C6 solids) for combined heat and power for the sugar mill, ethanol plant, and butanol plant (utilizing the remainder of the readily available source for energy for combined heat and power operation of the sugar mill). The use of ethanol vinasse for pretreatment water, wash water for sugar recovery from the liquid solids separation process, fermentation dilution water, nutrient addition, and vent gas scrubbing will reduce the overall requirement for water for the butanol plant; the need or demands for a water supply (i.e. well, river water, lake water etc.) is reduced. Molasses is available to provide organism nutrition for butanol production, reducing the potential need for additional supply of nutrients for organism nutrition (and helping to minimize the overall cost of production).

The following examples are intended to illustrate, but not limit, the invention.

### EXAMPLES

### Example 1

Experimental work was conducted to verify that bagasse from a sugar mill could be used as biomass for the production of butanol.

Compositional analysis of a sample of sugar cane bagasse is shown in Table 1.

TABLE 1

| | Measured | Range (+/−) |
|---|---|---|
| | (% by | (% by |
| Component | weight) | weight) |
| Ash | 4.0 | 1.0 |
| Water Extractives | 3.6 | 1.2 |
| Ethanol Extractives | 1.5 | 0.6 |
| Cellulose | 36.9 | 4.0 |
| Hemicellulose | 23.9 | 2.2 |
| Acid Soluble Lignin | 5.0 | 3.4 |
| Acid Insoluble Lignin | 18.4 | 1.5 |
| Total Lignin | 23.4 | 4.9 |
| Protein | 1.0 | 0.6 |
| Furans | 1.2 | 0.0 |
| Acetic Acid | 4.4 | 0.5 |

Estimated results from the use of bagasse in the production of sugars is shown in Table 2.

TABLE 2

| Weight | |
|---|---|
| (kg) | |
| 429.8 | Bagasse dry weight (est.) |
| 102.7 | Potential C5 production (100% yield) (est.) |
| 158.6 | Potential C6 production (100% yield) (est.) |
| 261.3 | Potential total sugar production (100% yield) (est.) |

Bagasse from two different sources was pretreated with nitric acid for hemicellulose extraction. 85 g of acid was used per kilogram of bagasse, accounting for buffering capacity of the bagasse and the water stream available from city water. A chip refiner (Andritz Fiber Refiner 401) was utilized to mix the acid, water, and bagasse. The plate spacing was set wide open to minimize any milling action on the bagasse while providing sufficient mixing. The machine output was to 55 gallon drums.

The acid impregnated material was elevated via a barrel lift that carried the drums to a plug screw feeder at the top of the digester. The feeder maintained a plug at its output that held the pressure and temperature of the subsequent plug flow reactor constant. The plug flow reactor temperature was set by adjusting the steam pressure (direct injection of steam) to 25-30 psi (pounds per square inch), providing a reaction temperature of 130° C. The reactor retention time was set to 35 minutes by adjusting the screw conveyer speed in the reactor. The liquid was separated from the residual material through the use of an Andritz model 560 screw press with an 8:1 compression ratio. Some small quantities of analytical samples were separated from the solids via filtration and/or centrifugation.

The composition of the liquid hydrolysate obtained is shown in Table 3.

TABLE 3

| | |
|---|---|
| Sample 1 | Sample 2 |

| | |
|---|---|
| Component | Concentration (g/L) |

| | | |
|---|---|---|
| Glucose | 6 | 5 |
| Xylose/mannose/galactose (XMG) | 57 | 61 |
| Arabinose | 6 | 8 |
| Lactic Acid | 3.11 | 1.22 |
| Glycerol | 0.00 | 0.38 |
| Formic Acid | 0.55 | 0.71 |
| Acetic Acid | 4.84 | 4.68 |
| Levulinic Acid | 0.19 | 0.47 |
| HMF | 0.13 | 0.18 |
| Furfural | 0.10 | 0.09 |
| Total monomeric sugars | 69 | 74 |

The data in Table 3 is for two separate batch runs of sugar cane bagasse. Sample 1 was bagasse collected from Louisiana (North America) and was stored in piles protected from rain and allowed to reach equilibrium with the ambient conditions over a period greater than 12 months. Sample 2 was bagasse collected from an unprotected storage pile at a sugar mill site in Brazil (South America) and transported in drums to the test site. The approximate time period from harvest to usage was estimated to be less than two months.

Samples were analyzed by HPLC using a procedure based on National Renewable Energy Laboratory Technical Report NREL/TP-510-42623 (January, 2008). Compositional analysis included monomeric sugars, organic acids, glycerol, hydroxymethyl furfural (HMF), and furfural. Generally, lactic and acetic acids do not inhibit the fermentation process at levels normally found in the biomass hydrolysates. The hemicellulose contains acetyl groups that become acetic acid upon hydrolysis. Lactic acid is usually the product of fermentation by a lacto bacillus. For sugar cane bagasse, the bagasse piles that the samples were taken from can be ensiling (fermenting) and producing lactic and acetic acid. Levulinic acid and HMF are degradation products of glucose. Levulinic acid may inhibit the fermentation process and the amount in Sample No. 2 may provide low levels of inhibition to microorganisms used for butanol fermentation. Formic acid and furfural are degradation products of xylose. Formic acid is or can be an inhibitory compound to the fermentation, while furfural is only inhibitory at higher concentrations.

The process outputs are shown in Table 4.

TABLE 4

| Potential total sugar production | | | |
|---|---|---|---|
| 216.9 kg | Sample 1 | Sample 2 | Average |
| Sugar produced (kg) | 70.0 | 74.1 | 72.0 |
| Sugar produced (% of total) | 32.3 | 34.1 | 33.2 |
| Glucose produced (kg) | 6.0 | 5.0 | 5.5 |
| Xylose and arabinose produced (kg) | 64.0 | 66.0 | 65.0 |
| Non-sucrose oligomers produced (kg) | 1.3 | 2.2 | 1.8 |
| Sugar produced (kg) per kg of bagasse | 0.1 | 0.1 | 0.1 |
| processed | | | |
| % Discharge solids (w/w) | 14.2 | 17.0 | 15.6 |

These sugar streams were fermented at 45 to 50 g/L sugar, without procedures for removal of inhibitors. The pH of liquid hydrolysate from each of the runs was adjusted to 6.5-7. Corn steep powder (7 g/l), and trace salts (magnesium sulfate, manganese sulfate, ferrous sulfate, and citric acid monohydrate) were added to the hydrolysate. A butanol-producing Clostridium strain from a seed train grown in yeast extract medium (YEM) was inoculated into 4 ml of hydrolysate at a 1:10 dilution and fermented anaerobically for 72 hours. Fermentations were carried out in an anaerobic hood at a temperature of about 30° C., and butanol was produced from both hydrolysate samples.

### Example 2

Experimental work was conducted to determine if ethanol vinasse would perform suitably as a primary or supplemental water source for a fermentation process.

Media including acid-hydrolyzed sugar cane bagasse (“bagasse media”) was formulated using vinasse to dilute the sugar concentration to levels suitable for fermentation. The vinasse used in this example was produced from a commercial ethanol fermentation process using cane sugar substrate and a yeast biocatalyst, and recovered as the bottom fraction of ethanol distillation from the fermentation broth.

The bagasse media included bagasse hydrolysate, molasses, yeast extract, ammonium sulfate, potassium phosphate buffer, trace elements and 0 to 33% (v/v) vinasse. One further media formulation was prepared that included only bagasse hydrolyste and vinasse (44% vinasse) and no other media additive.

Cultures of a solvent-producing Clostridium strain were prepared with a 10% inoculum in 4 ml media in grown in 15 ml conical tubes. The cultures were incubated in an anaerobic chamber at 30° C. for 48 hours. The results are shown in Table 5. Vinasse had no negative effect on fermentation performance. Vinasse also supported butanol production in the bagasse media with no other additives.

TABLE 5

| | Butanol | | |
|---|---|---|---|
| | Titer | | |
| | (g/l) | Sugar | Butanol |
| | (% Relative | Conversion | Yield |
| Medium | to Control) | (%) | (g/g) |
| Control (no vinasse) | 100 | 93 | 0.32 |
| 3% Vinasse | 100.9 | 93 | 0.32 |
| 11% Vinasse | 100.9 | 94 | 0.32 |
| 33% Vinasse | 104.3 | 92 | 0.32 |
| Hydrolysate + vinasse without | 25.2 | 32 | 0.24 |
| additional nutrients (44% vinasse) | | | |

### Example 3

Media including sugar cane molasses (“molassses media”) was formulated using vinasse prepared as described in Example 2 to dilute the sugar concentration to levels suitable for fermentation. The molasses media included molasses, yeast extract, ammonium acetate, and 0 to 85% (v/v) vinasse.

Cultures of a solvent-producing Clostridium strain were prepared with a 10% inoculum in 4 ml media in grown in 15 ml conical tubes. The cultures were incubated in an anaerobic chamber at 30° C. for 48 hours. The results are shown in Table 6. Vinasse had no negative effect on fermentation performance.

TABLE 6

| | Butanol Titer | | |
|---|---|---|---|
| | (g/l) | Sugar | Butanol |
| | (% Relative to | Conversion | Yield |
| Medium | Control) | (%) | (g/g) |
| Control (no vinasse) | 100 | 77 | 0.30 |
| 22.5% Vinasse | 103.1 | 78 | 0.31 |
| 45% Vinasse | 103.1 | 79 | 0.29 |
| 62.5% Vinasse | 104.6 | 87 | 0.27 |
| 85% Vinasse | 101.5 | 76 | 0.30 |

### Example 4

50 g dry weight bagasse samples were hydrolyzed in a mixture that contained 2 g 70% nitric acid, 1.94 g crude glycerol, varying concentrations of yeast cells 0 g/kg, 0.5 g/kg, 1.0 g/kg, 1.5 g/Kg, 2.0 g/kg, and 2.5 g/kg, to a final reactor weight of 350 g. Liquid hydrolysate was separated from residual solid material. The pH was adjusted to 6.8 to 7.0 with NaOH and sterilized. Cultures of a solvent-producing Clostridium strain were prepared with a 10% inoculum in 4 ml media (no yeast extract added) grown in 15 ml conical tubes. The cultures were incubated in an anaerobic chamber at 32° C. for 72 hours. Butanol concentrations were determined by HPLC analysis. Control without yeast was used for comparison and set at 100%. Yeast cells added prior to hydrolysis resulted in a 128-134% improvement in butanol production compared to the control with no yeast cells added.

TABLE 7
Percent Improvement in Butanol Production with Yeast Cell Addition
Prior to Biomass Hydrolysis

| Amount | No Added yeast |
|---|---|
| of Yeast (g/kg)* | cells |
| 0 | 100% |
| 0.5 | 134% |
| 1.0 | 133% |
| 1.5 | 134% |
| 2.0 | 128% |
| 2.5 | 130% |

*Added prior to hydrolysis

### Example 5

Spent yeast cells, obtained from a sugar cane mill ethanol fermentation, were hydrolyzed by heat treatment. Hydrolyzed yeast were added as a nutrient to a sugar mixture at 0, 2, and 4 g/L concentration and compared to controls with Beckton Dickson Bacto yeast extract. Cultures of a solvent-producing Clostridium strain were prepared with a 10% inoculum in 4 ml media grown in 15 ml conical tubes. The cultures were incubated in an anaerobic chamber at 32° C. for 72 hours. Control with no yeast was used for comparison. Hydrolyzed spent yeast cells at 2 and 4 g/L from ethanol fermentation were able to serve as a nutrient in bacterial fermentation, improving butanol production by 108% and 131%, respectively. Hydrolyzed spent yeast cells at 4 g/L was similar to the performance with 2 g/L of BD Bacto yeast extract.

TABLE 8
Butanol production with hydrolyzed spent yeast cells compared to BD
Bacto yeast extract

| | | |
|---|---|---|
| | | BD Bacto |
| Amount of | Spent Yeast | Yeast |
| Yeast | Cells | Extract |

| | |
|---|---|
| 0 | 100% |

| | | |
|---|---|---|
| 2 | 108% | 139% |
| 4 | 131% | 156% |

### Example 6

Media including sugar cane molasses (“molassses media”) was formulated using butanol vinasse prepared from a demonstration scale butanol fermentation process and recovered as the bottom fraction of the butanol distillation from the fermentation broth. The molasses media included molasses, yeast extract, ammonium acetate, and 0 to 77% (v/v) butanol vinasse. Cultures of a solvent-producing Clostridium strain were prepared with a 10% inoculum in 4 ml media in grown in 15 ml conical tubes. The cultures were incubated in an anaerobic chamber at 30° C. for 48 hours. The results are shown in Table 9. Utilization of butanol vinasse at 38% and 77% (v/v) for preparation of the fermentation media improved butanol production by 105% and 120%, respectively.

TABLE 9

| Medium | % Butanol vs Control |
|---|---|
| Control (process water, no vinasse) | 100% |
| Process water and 38% vinasse | 105% |
| Process water and 77% vinasse | 120% |

### Example 7

Cane straw was pretreated using nitric acid for hemicellulose extraction. A coffee grinder was used to mill the straw to <2 mm and nitric acid and water were mixed with the ground biomass in a beaker. Thirty five milligrams of nitric acid was dosed to one gram of straw (dry matter). The final ratio of water to dry solids in the mixture was 4:1. Six grams of this mixture was added to a 316 stainless steel tube reactor (½″ diameter×6″ length) and the reactor was sealed with stainless steel caps on both ends. Next, the sealed tube was heated in a fluidized sand bath reactor at 145° C. for a residence time of 35 minutes (plus 2 minute heat-up time). After the heat treatment, the tube was quenched in cool water, and the treated material was unloaded and HPLC samples were taken for analysis. Table 10 shows the cane straw solids composition and Table 11 shows the composition of the hydrolysate.

TABLE 10
Cane Straw Composition (% weight of Solids)

| | Measured | Range (+/−) |
|---|---|---|
| Component | (%) | (%) |
| Ash | 8.2 | 4 |
| Cellulose | 40.7 | 4 |
| Hemicellulose | 22.3 | 3 |
| Acid Soluble Lignin | 0.7 | 0.3 |
| Acid Insoluble Lignin | 20.1 | 4 |
| Total Lignin | 20.8 | 4.3 |

TABLE 11
Composition of Cane Straw Hydrolysate

| | Concentration |
|---|---|
| Component | (g/L) |
| Glucose | 9 |
| Xylose/mannose/galactose (XMG) | 50 |
| Arabinose | 8 |
| Formic Acid | 0.9 |
| Acetic Acid | 4.2 |
| Levulinic Acid | 0.2 |
| HMF | 0.45 |
| Furfural | 1.5 |
| Total monomeric sugars | 69 |

### Example 8

Bagasse was pretreated using nitric acid for hemicellulose extraction. Bagasse was dried in a 50° C. oven to a dry matter of around 90%. A coffee grinder was used to mill the bagasse to <2 mm and nitric acid and water were mixed with the ground bagasse in a beaker. Twenty five milligrams of nitric acid was dosed to one gram of bagasse (dry matter). The final ratio of water to dry solids in the mixture was 4:1. Six grams of this mixture was added to a 316 stainless steel tube reactor (½″ diameter×6″ length) and the reactor was sealed with stainless steel caps on both ends. Next, the sealed tube was heated in a fluidized sand bath reactor at 145° C. for a residence time of 45 minutes (plus 2 minute heat-up time). After the heat treatment, the tube was quenched in cool water, and the treated material was unloaded and HPLC samples were taken for analysis. Table 12 shows the composition of the hydrolysate.

TABLE 12
Composition of bagasse hydrolysate

| Component | Concentration (g/L) |
|---|---|
| Glucose | 4.3 |
| Xylose/mannose/galactose (XMG) | 41.1 |
| Arabinose | 3.3 |
| Formic Acid | 0.5 |
| Acetic Acid | 7.5 |
| Levulinic Acid | 0 |
| HMF | 0.1 |
| Furfural | 2.6 |
| Total monomeric sugars | 48.7 |

### Example 9

Brazilian sugarcane bagasse was pretreated with nitric acid to extract hemicellulose sugars. This material was fed to a three stage countercurrent wash system using a 1.5:1 vinasse:solids ratio. Butanol vinasse was heated to 90° C. and then used in the wash system. The final liquid output recovered about 90% of the available soluble sugars from the initial material.

### Example 10

1200 g of Brazilian sugarcane bagasse was pretreated with nitric acid to extract hemicellulose. The initial hydrolysate was squeezed with a screw press to a solids level of 50% solids (50% moisture).

A 200 g sample was removed and analyzed for nitrates using EPA method 353.2. 750 g H₂O was added and mixed, then the sample was again squeezed to 50% moisture with a screw press. A total of four washes were performed in this manner, using a 1.5:1 wash water:solids weight ratio for each wash. The results are shown in Table 13.

TABLE 13

| No. of Washes | Nitrates in Solids (mg/kg) |
|---|---|
| 0 | 686 |
| 1 | 354 |
| 2 | 192 |
| 3 | 98 |
| 4 | 55 |

### Example 11

Sugar cane bagasse is hydrolyzed with nitric acid as described in Example 1. Composition of hydrolysate according to an exemplary embodiment is shown in Table 14.

TABLE 14

| Parameter | Units | Typical Range |
|---|---|---|
| pH | — | 1-2 |
| Crude Protein | g/L | 9-13 |
| Total Sugars | g/L | 70-80 |
| Glycerol | g/L | 0-10 |
| Acetic acid | g/L | 4-6 |
| Formic acid | g/L | 0.1-1 |
| Lactic acid | g/L | 1-2 |
| Yeast | g/L | 0-5 |
| Calcium | mg/L | 100-200 |
| Copper | mg/L | 1-3 |
| Iron | mg/L | 500-800 |
| Magnesium | mg/L | 50-200 |
| Manganese | mg/L | 5-20 |
| Phosphorus | mg/L | 10-50 |
| Sulfur | mg/L | 50-150 |
| Zinc | mg/L | 1-2 |

Composition of residual solids following separation of liquid hydrolysate from residual solids according to an exemplary embodiment is shown in Table 15.

TABLE 15

| Component | Wt % | Range (+/−) |
|---|---|---|
| Glucan | 55.49% | 2% |
| Lignin | 33.84% | 1% |
| Ash | 4.03% | 1% |
| Xylan | 3.49% | 0.50% |
| Uronics | 2.75% | 0.50% |
| Arabinan | 0.25% | 0.10% |
| Galactan | 0.14% | 0.01% |
| Biomass | 0.02% | 0.005% |
| % moisture | 55.00% | 5% |

### Example 12

Ethanol vinasse is prepared from ethanol-containing fermentation broth. Composition of ethanol vinasse according to an exemplary embodiment is shown in Table 16.

TABLE 16

| Parameter | Units | Expected Range | Typical Range |
|---|---|---|---|
| pH | — | 3.5-5 | 4-4.5 |
| Dry Matter | g/L | 11-39 | 22-28 |
| Crude | g/L | 1-5 | 2-3 |
| Protein | | | |
| Total Sugars | g/L | 1-5 | 2-3 |
| Glycerol | g/L | 2-30 | 4-6 |
| Ethanol | mg/L | 100-5000 | 600-800 |
| Acetic acid | mg/L | 100-1000 | 400-500 |
| Formic acid | mg/L | 100-1000 | 200-300 |
| Lactic acid | mg/L | 100-1000 | 600-700 |
| Yeast | mg/L | 100-1500 | 300-500 |
| Calcium | mg/L | 100-1000 | 500-600 |
| Chloride | mg/L | 500-2300 | 1100-1300 |
| Copper | mg/L | 0.5-3 | 1-1.5 |
| Iron | mg/L | 2-200 | 20-30 |
| Magnesium | mg/L | 100-500 | 200-250 |
| Manganese | mg/L | 1-12 | 4-5 |
| Nitrogen | mg/L | 100-1000 | 300-400 |
| Phosphorus | mg/L | 10-200 | 20-100 |
| Potassium | mg/L | 800-4000 | 1800-2200 |
| Sodium | mg/L | 10-250 | 40-60 |
| Sulfur | mg/L | 800-3000 | 1400-1700 |
| Zinc | mg/L | 0.5-5 | 1-2 |

It is important to note that the construction and arrangement of the elements of the embodiments of inventions as described in this application and as shown in the figures herein is illustrative only. Although some embodiments of the present inventions have been described in detail in this disclosure, those skilled in the art who review this disclosure will readily appreciate that many modifications are possible without materially departing from the novel teachings and advantages of the subject matter recited. Accordingly, all such modifications are intended to be included within the scope of the present inventions. Other substitutions, modifications, changes and omissions may be made in the design, operating conditions and arrangement of the preferred and other exemplary embodiments without departing from the spirit of the present inventions.

It is important to note that the systems and methods of the present inventions can comprise conventional technology (e.g., biomass processing, biofuel production, product recovery, etc.) or any other applicable technology (present or future) that has the capability to perform the functions and processes/operations indicated in the FIGURES. All such technology is considered to be within the scope of the present inventions.

We claim:

1. An integrated biorefinery, comprising:
(a) a sugar production facility, wherein sugar-containing biomass is processed to extract sugar, thereby producing a liquid sugar-containing extract and residual bagasse; and
(b) a bioproduct production facility, comprising a microorganism that is capable of producing at least one bioproduct in a microbial fermentation process,
wherein sugar molecules that are extracted from sugar-containing biomass that is processed in the sugar production facility are provided to the microorganism in a growth medium,
wherein the sugar molecules are fermented by the microorganism, thereby producing a fermentation broth that comprises said at least one bioproduct.

2. An integrated biorefinery according to claim 1, wherein the sugar molecules that are provided to the microorganism comprise: (i) at least a portion of the liquid sugar-containing extract, and (ii) sugar molecules extracted from at least a portion of the residual bagasse and/or sugar molecules extracted from at least a portion of biomass that is removed from the sugar-containing biomass prior to processing in the sugar production facility.

3. An integrated biorefinery according to claim 1, wherein said sugar-containing biomass comprises sugar cane.

4. An integrated biorefinery according to claim 1, wherein said sugar-production facility comprises a sugar mill.

5. An integrated biorefinery according to claim 2, wherein said sugar-containing biomass comprises sugar cane, and wherein said biomass that is removed prior to processing in the sugar production facility comprises cane straw.

6. An integrated biorefinery according to claim 1, wherein said at least one bioproduct is separated from the fermentation broth, thereby producing vinasse, and
wherein at least a portion of the vinasse is recycled and provided to the bioproduct production facility as liquid in the growth medium for further production of said at least one bioproduct.

7. An integrated biorefinery according to claim 1, wherein said at least one bioproduct comprises butanol and/or acetone.

8. An integrated biorefinery according to claim 6, wherein said at least one bioproduct comprises butanol, and wherein said vinasse is butanol vinasse.

9. An integrated biorefinery according to claim 1, wherein said processing of sugar-containing biomass in the sugar production facility comprises steam, wherein a steam condensate is produced from said steam, and wherein at least a portion of said steam condensate is provided to the bioproduct production facility as liquid in the growth medium for production of the bioproduct.

10. An integrated biorefinery according to claim 1, wherein said processing of sugar-containing biomass in the sugar production facility comprises steam, wherein at least a portion of said steam is recovered and used to provide heat for the fermentation process in the bioproduct production facility.

11. An integrated biorefinery according to claim 2, wherein the liquid sugar-containing extract comprises cane juice and/or molasses.

12. An integrated biorefinery according to claim 2, wherein sugar molecules are extracted from at least a portion of the bagasse and/or from biomass removed prior to processing in the sugar production facility by acid hydrolysis, thereby producing a liquid hydrolysate that comprises soluble sugar molecules and residual solid material.

13. An integrated biorefinery according to claim 12, wherein acid hydrolysis comprises hydrolysis with nitric acid.

14. An integrated biorefinery according to claim 12, wherein said hydrolysate comprises C5 sugar molecules.

15. An integrated biorefinery according to claim 12, further comprising a boiler, wherein said residual solid material is provided to the boiler as a fuel source.

16. An integrated biorefinery according to claim 15, wherein said residual solid material comprises nitrates, and wherein at least a portion of said nitrates are removed prior to providing the material to the boiler.

17. An integrated biorefinery according to claim 12, wherein said residual solid material comprises cellulose.

18. An integrated biorefinery according to claim 17, wherein said residual solid material is treated to extract sugar molecules from said cellulose.

19. An integrated biorefinery according to claim 18, wherein extraction of sugar molecules from cellulose comprises treatment with at least one enzyme that catalyzes hydrolysis of cellulose, thereby producing a liquid enzymatic hydrolysate that comprises soluble sugar molecules and a second residual solid material.

20. An integrated biorefinery according to claim 19, further comprising a boiler, wherein said second residual solid material is provided to the boiler as a fuel source.

21. An integrated biorefinery according to claim 20, wherein said second residual solid material comprises nitrates, and wherein at least a portion of said nitrates are removed prior to providing the material to the boiler.

22. An integrated biorefinery according to claim 1, wherein the microorganism in the bioproduct production facility comprises a Clostridium strain.

23. An integrated biorefinery according to claim 1, further comprising:
(c) an ethanol production facility, comprising a second microorganism that is capable of producing ethanol in a second microbial fermentation process,
wherein sugar molecules that are extracted from sugar-containing biomass that is processed in the sugar production facility are provided to the second microorganism in a second growth medium, and
wherein the sugar molecules are fermented by the second microorganism, thereby producing a second fermentation broth that comprises ethanol.

24. An integrated biorefinery according to claim 23, wherein ethanol is separated from the second fermentation broth, thereby producing ethanol vinasse, and
wherein the ethanol vinasse is provided to the bioproduct production facility as liquid for the growth medium.

25. An integrated biorefinery according to claim 24, wherein the ethanol vinasse provides nutrients for growth of the microorganism in the bioproduct production facility.

26. An integrated biorefinery according to claim 23, wherein said second microorganism comprises yeast, and wherein at least a portion of the yeast from the ethanol production facility is provided to the bioproduct production facility in the growth medium for the microorganism that produces said at least one bioproduct, wherein said yeast provides nutrients for growth of said microorganism.

27. An integrated biorefinery according to claim 23, wherein said second microorganism comprises yeast, wherein at least a portion of the yeast from the ethanol production facility is added during acid hydrolysis of bagasse and/or biomass that is removed prior to sugar processing in the sugar production facility to produce a hydrolysate that comprises hydrolyzed yeast cells, wherein said hydrolysate is provided in the growth medium for the microorganism in the bioproduct production facility, and wherein said hydrolyzed yeast cells provide nutrients for growth of said microorganism.

28. An integrated biorefinery according to claim 1, wherein said microorganism is processed after said fermentation and incorporated into an animal feed product.

29. A process for producing a bioproduct, comprising culturing a microorganism that is capable of producing the bioproduct in a growth medium that comprises a liquid sugar-containing extract from processing of sugar-containing biomass and sugar molecules extracted from bagasse and/or biomass that is removed from sugar-containing biomass prior to processing in a sugar production facility.

30. A process according to claim 29, wherein the liquid sugar-containing extract comprises cane juice and/or molasses.

31. A process according to claim 29, wherein said sugar-containing biomass comprises sugar cane.

32. A process according to claim 29, wherein said biomass that is removed prior to processing in the sugar production facility comprises cane straw.

33. A process according to claim 29, wherein said at least one solvent comprises butanol and/or acetone.

34. A process for producing at least one bioproduct, comprising:
(a) processing sugar-containing biomass in a sugar production facility, thereby producing a liquid sugar-containing extract and residual bagasse; and
(b) culturing a microorganism in a bioproduct production facility, wherein sugar molecules that are extracted from sugar-containing biomass that is processed in the sugar production facility are provided to the microorganism in a growth medium, wherein the sugar molecules are fermented by the microorganism, thereby producing a fermentation broth that comprises said at least one bioproduct.

35. A process according to claim 34, wherein the sugar molecules that are provided to the growth medium comprise: (i) at least a portion of the liquid sugar-containing extract; and (ii) sugar molecules extracted from at least a portion of the residual bagasse and/or sugar molecules extracted from at least a portion of biomass that is removed from the sugar-containing biomass prior to processing in the sugar production facility.

36. A process according to claim 34, wherein said sugar-containing biomass comprises sugar cane.

37. A process according to claim 34, wherein said sugar-production facility comprises a sugar mill.

38. A process according to claim 35, wherein said sugar-containing biomass comprises sugar cane, and wherein said biomass that is removed prior to processing in the sugar production facility comprises cane straw.

39. A process according to claim 34, wherein said at least one bioproduct is separated from the fermentation broth, thereby producing vinasse, and
wherein at least a portion of the vinasse is recycled and provided to the bioproduct production facility as liquid in the growth medium for further production of said at least one bioproduct.

40. A process according to claim 34, wherein said at least one bioproduct comprises butanol and/or acetone.

41. A process according to claim 39, wherein said at least one bioproduct comprises butanol, and wherein said vinasse is butanol vinasse.

42. A process according to claim 34, wherein said processing of sugar-containing biomass in the sugar production facility comprises steam, wherein at least a portion of said steam is condensed to produce a steam condensate, and wherein at least a portion of said steam condensate is provided as liquid in the growth medium for production of the bioproduct.

43. A process according to claim 34, wherein said processing of sugar-containing biomass in the sugar production facility comprises steam, wherein at least a portion of said steam is recovered and used to provide heat for the fermentation.

44. A process according to claim 34, wherein the liquid sugar-containing extract comprises cane juice and/or molasses.

45. A process according to claim 34, wherein sugar molecules are extracted from at least a portion of the bagasse and/or from biomass removed prior to processing in the sugar production facility by acid hydrolysis, thereby producing a liquid hydrolysate that comprises soluble sugar molecules and residual solid material.

46. A process according to claim 45, wherein acid hydrolysis comprises hydrolysis with nitric acid.

47. A process according to claim 46, wherein said liquid hydrolysate comprises C5 sugar molecules.

48. A process according to claim 45, further comprising a boiler, wherein said residual solid material is separated from the liquid hydrolysate, and wherein the residual solid material is provided to the boiler as a fuel source.

49. A process according to claim 48, wherein said residual solid material comprises nitrates, and wherein at least a portion of said nitrates are removed prior to providing the material to the boiler.

50. A process according to claim 45, wherein said residual solid material comprises cellulose.

51. A process according to claim 45, wherein said residual solid material is separated from the liquid hydrolysate, and wherein said residual solid material is treated to extract sugar molecules from said cellulose.

52. A process biorefinery according to claim 51, wherein extraction of sugar molecules from cellulose comprises treatment with at least one enzyme that that catalyzes hydrolysis of cellulose, thereby producing a liquid enzymatic hydrolysate that comprises soluble sugar molecules and a second residual solid material.

53. A process according to claim 52, further comprising a boiler, wherein said second residual solid material is separated from the liquid enzymatic hydrolysate, and wherein the second residual material is provided to the boiler as a fuel source.

54. A process according to claim 53, wherein said second residual solid material comprises nitrates, and wherein at least a portion of said nitrates are removed prior to providing the material to the boiler.

55. A process according to claim 34, wherein the microorganism comprises a Clostridium strain.

56. A process according to claim 34, further comprising:
(c) producing ethanol in an ethanol production facility, wherein the ethanol production facility comprises a second microorganism that is capable of producing ethanol in a second microbial fermentation process,
wherein sugar molecules that are extracted from sugar-containing biomass that is processed in the sugar production facility are provided to the second microorganism in a second growth medium, and
wherein the sugar molecules are fermented by the second microorganism, thereby producing a second fermentation broth that comprises ethanol.

57. A process according to claim 56, wherein ethanol is separated from the second fermentation broth, thereby producing ethanol vinasse, and
wherein the ethanol vinasse is provided as liquid to the culture medium in the bioproduct production facility.

58. A process according to claim 57, wherein the ethanol vinasse provides nutrients for growth of the microorganism in the bioproduct production facility.

59. A process according to claim 56, wherein said second microorganism comprises yeast, and wherein at least a portion of the yeast from the ethanol production facility is provided to the culture medium in the bioproduct production plant as nutrition for the microorganism that produces said at least one bioproduct.

60. A process according to claim 56, wherein said second microorganism comprises yeast, wherein at least a portion of the yeast from the ethanol production plant is added during acid hydrolysis of bagasse and/or biomass that is removed prior to sugar processing in the sugar production facility to produce a hydrolysate that comprises hydrolyzed yeast cells, wherein said hydrolysate is provided in the growth medium for the microorganism in the bioproduct production facility, and wherein said hydrolyzed yeast cells provide nutrients for growth of said microorganism.

61. A process according to claim 34, wherein said microorganism is processed after said fermentation and incorporated into an animal feed product.

62. A growth medium for culturing a microorganism, comprising: (a) a liquid sugar-containing extract from processing of sugar-containing biomass; and (b) sugar molecules extracted from bagasse and/or biomass that is removed from sugar-containing biomass prior to processing in a sugar production facility.

63. A growth medium according to claim 62, wherein said liquid sugar-containing extract comprises cane juice and/or molasses.

64. A growth medium according to claim 62, comprising both sugar molecules extracted from bagasse and sugar molecules extracted from biomass that is removed from sugar-containing biomass prior to processing in a sugar production facility.

65. A growth medium according to claim 62, wherein said sugar-containing biomass comprises sugar cane.

66. A growth medium according to claim 65, wherein said biomass that is removed from sugar-containing biomass prior to processing in a sugar production facility comprises cane straw.

67. A growth medium according to claim 62, further comprising vinasse.

68. A growth medium according to claim 62, further comprising steam condensate from a sugar production facility.

69. A growth medium according to claim 62, further comprising hydrolyzed and/or lysed yeast cells.
